(19) United States
(12) Patent Application Publication
(10) Pub. No.: US 2023/0042960 A1
(43) Pub. Date: Feb. 9, 2023

(54) MICROBIAL COMPOSITIONS AND METHODS FOR TREATMENT AND DETECTION OF DISEASE

(71) Applicant: Northeastern University, Boston, MA (US)

(72) Inventors: Kim LEWIS, Newton, MA (US); Madeleine MORRISSETTE, Arlington, MA (US); Philip STRANDWITZ, Medford, MA (US); Anthony D'ONOFRIO, Northborough, MA (US); Norman PITT, Jamaica Plain, MA (US)

(73) Assignee: Northeastern University, Boston, MA (US)

(21) Appl. No.: 17/784,162

(22) PCT Filed: Dec. 10, 2020
(86) PCT No.: PCT/US2020/064284

§ 371 Date: Jun. 10, 2022

Related U.S. Application Data
(60) Provisional application No. 63/035,102, filed on Jun. 5, 2020.
(60) Provisional application No. 63/013,796, filed on Apr. 22, 2020.
(60) Provisional application No. 62/945,984, filed on Dec. 10, 2019.

(51) Int. Cl. A61K 35/741; A61K 35/745; C12Q 1/06; C12Q 1/689; A61P 1/00

(52) U.S. Cl. CPC A61K 35/741; A61K 35/745; C12Q 1/06; C12Q 1/689; A61P 1/00

(57) ABSTRACT

Disclosed herein are compositions and methods for the treatment and/or detection of diseases such as, but not limited to Lyme disease, Lyme disease-related disorders including post-treatment Lyme disease syndrome (PTLDS), chronic Lyme disease (CLD) and/or inflammation.

### CROSS-REFERENCE TO RELATED APPLICATIONS

This application claims priority to U.S. Ser. No. 62/945,984, filed on Dec. 10, 2019 and entitled Modulation of the Gut Microbiome for Treatment of Inflammatory Bowel Disease; U.S. Ser. No. 63/035,102, filed on Jun. 5, 2020 and entitled Modulation of the Gut Microbiome for Treatment of Disease; and U.S. Ser. No. 63/013,796, filed on Apr. 22, 2020 and entitled Using the Fecal Microbiome Composition as a Diagnostic Tool for post-treatment Lyme disease syndrome, the contents of each of which are herein incorporated by reference in their entireties.

### FIELD OF THE DISCLOSURE

The present disclosure relates to compositions and methods for treating and/or diagnosing disease conditions associated with inflammation such as, but not limited to, Lyme disease, post-treatment Lyme disease syndrome, inflammatory bowel disease and/or colitis. In particular, the present disclosure utilizes bacterial species isolated from and/or associated with the microbiome of a subject to identify therapeutic strategies and design disease detection tools.

### BACKGROUND OF THE DISCLOSURE

The microbiome, in particular the gut microbiome of an individual, plays an important role in human health and has been shown to strongly influence host metabolism, the immune system, and the nervous system, also providing crucial colonization resistance against a range of intestinal pathogens. Microbiome compositional changes can alter immune tolerance. For instance, members of the intestinal microbiome have been characterized as contributing to the development of the long-term sequelae of acute infection events upon disruption of tissue and immune homeostasis. Studies have found the microbiome to be on par with and often superior to the human genome in predicting disease states. Indeed, many microbiome-wide association studies have established correlation, and sometimes causation, of the gut microbiome in diseases such as multiple sclerosis, rheumatoid arthritis, and systemic lupus.

Inflammatory bowel disease (IBD) is a chronic immune-mediated disease affecting the gastrointestinal tract. The disease is thought to develop as a result of the interactions between environmental, microbial, and immune-mediated factors in a genetically susceptible host. IBD may also be fueled by an increase in Enterobacteriaceae, which disrupts the gut microbiome. Currently, there are no probiotics or live bacterial formulations, particularly those targeting Enterobacteriaceae, that have been shown to be effective in treating IBD.

The microbiome has also been implicated in many diseases with symptoms that overlap those of post-treatment Lyme disease, including autoimmune diseases. Lyme disease, caused by Borrelia burgdorferi, is the most common vector-borne illness in the United States, affecting approximately 300,000 Americans per year. Acute Lyme disease is a multi-systemic disease that presents with flu-like symptoms and can cause arthritis, meningitis, cranial nerve palsy, radicular pains and/or carditis. Antibiotic treatment typically cures Lyme disease; however, about 10-20% of patients treated for Lyme disease experience persistent symptoms including fatigue, arthralgia, myalgia, and mood or memory disturbances. These symptoms can last months to years after treatment and, when accompanied by functional impairment, are collectively referred to as post-treatment Lyme disease syndrome (PTLDS). Estimates for the number of patients with persistent symptoms after treatment are 40,000-80,000 people each year. There remains a need to identify therapeutic agents that may be useful in the treatment of Lyme disease and PTLDS in particular and/or therapeutic agents that may be useful in the treatment of the early phase of the disease, thereby preventing the onset of PTLDS. Objective diagnostic tools for detection of Lyme disease are also currently lacking and current diagnosis of Lyme disease relies predominantly on the results of a clinical exam and a history of exposure to Lyme-endemic areas.

### SUMMARY OF THE DISCLOSURE

The present disclosure describes, inter alia, compositions for inhibiting the growth of at least one species of Enterobacteriaceae. As a non-limiting example, the species of Enterobacteriaceae may Escherichia coli. The compositions may include or consist essentially of at least one bacterial species. Non-limiting examples of bacterial species present in the compositions of the disclosure include, but are not limited to, Gordonibacter pamelaeae, Clostridium bifermentans, Veillonella ratti, Paraclostridium benzoelyticum, Sutterella wadsworthia, Alisteps onderdonkii, Barnesiella intestinihominis, Clostridium hathewayi, Bifidobacterium catenulatum, Anaerinibacillus anaerinlyticus, Coprobacillus catenformis, and/or Coprococcus comes.

The compositions of the disclosure may include or consist essentially of a bacterial population of three bacterial species. In some embodiments, the three bacterial species may be Gordonibacter pamelaeae, Clostridium bifermentans, and Veillonella ratti. The ratio of the three bacterial species present in the bacterial population may be, e.g., 2:1:1 or 1:1:1. The composition may include or consist essentially of at least 1×10⁸ colony-forming units (CFU) of the bacterial population. In some embodiments, the compositions may inhibit the growth of at least one species of Entereobacteriaceae by about 10-20 fold. As a non-limiting example, the composition may inhibit the growth of the Enterobacteriaceae by 14 fold.

In some embodiments, the compositions may include or consist essentially of a bacterial population of one species. For example, the composition may include or consist essentially of a bacterial population of Gordonibacter pamelaeae. Such compositions may inhibit the growth of Enterobacteriaceae by 10 fold. In one aspect, the composition includes or consists essentially of a bacterial population of Clostridium bifermentans. Compositions of C. bifermentans may inhibit the growth of Enterobacteriaceae by 5 fold. In one embodiment, the composition includes or consists essentially of a bacterial population of Veillonella ratti.

The present disclosure also describes pharmaceutical compositions including the compositions described herein and at least one physiologically suitable carrier.

Also described herein are methods of treating PTLDS that include contacting a subject with, or administering to the subject, the compositions or the pharmaceutical compositions described herein. Also described herein are methods of treating inflammation, preventing inflammation and/or improving the survival of a subject. Such methods may involve contacting a subject with, or administering to the subject, the compositions or pharmaceutical compositions described herein. In some embodiments, the inflammation may be colitis or IBD.

Compositions of the disclosure may be administered to a subject by an oral route, buccal route, a subcutaneous route, an intravenous route, an intramuscular route, an intraperitoneal route, a transdermal route, an ocular route, a vaginal route, a nasal route, and/or a topical route. Compositions of the present disclosure may be provided to the subject at doses effective in achieving the intended purpose. As a non-limiting example, the compositions of the disclosure may be administered at of 1×10⁸ CFU of the bacterial population/kg body weight of the subject.

The present disclosure describes methods of diagnosing PTLDS in a subject. The method of diagnosing may involve the steps of (i) obtaining a sample from the subject; (ii) measuring the relative abundance of one or more bacterial genera in the sample to prepare a microbiome signature; and (iii) comparing the microbiome signature of the sample to the microbiome signature of a healthy control cohort. A difference in the relative abundance of one or more bacterial genera in the microbiome signature of sample compared to the microbiome signature of the healthy control cohort confirms the presence of PTLDS in the subject. In some embodiments, the bacterial genera may be Blautia, Clostridium, Roseburia, Staphylococcus, Bacteroides Parabacteroides, Barnesiella, Faecalibacterium, Enterococcus, Escherichia, Akkermansia, Alistipes, Barnesiella, Bifidobacterium, Catenibacterium, Collinsella, Coprococcus, Dialister, Dorea, Eubacterium, Lactobacillus, Methanobrevibacter, Prevotella, Ruminococcus, Shigella, Streptococcus, and/or Subdoligranulum. The sample obtained from the subject may be a stool sample. Prior to measuring the levels of one or more bacterial genera, 16S rRNA is extracted from the sample. The levels of one or more bacterial genera in the sample may be measured by fecal or cecal 16S rDNA sequencing, shotgun metagenomic sequencing, or transcriptomics or comparable methods known in the art. Alternatively, the genes of Enterobactericeae involved in anaerobic respiration may be measured by qPCR, transcriptomics, proteomics, or comparable methods. Further references to 16S rDNA sequencing should be understood to encompass these various alternatives.

As a non-limiting example, the microbiome signature may include the bacterial genus, Blautia, Staphylococcus and/or Roseburia. In one embodiment, the species of Blautia may be Blautia obeum. In one embodiment, the species of Staphylococcus may be Staphylococcus may be Staphylococcus aureus. In one aspect, the relative abundance of the species described herein may be greater in the sample with PTLDS than in the healthy control cohort. In some embodiments, the relative abundance of Blautia may be about 5-10%. As a non-limiting example, the relative abundance of Blautia may be 8.86%. In some embodiments, the relative abundance of Staphylococcus may be about 0.001-0.1%. As a non-limiting example, the relative abundance of Staphylococcus may be 0.0024%. In some embodiments, the relative abundance of Roseburia may be about 0.1-0.2%. As a non-limiting example, the relative abundance of Roseburia may be 0.15%.

In some embodiments, the microbiome signature of the present disclosure may include one or more Operational Taxonomic Units (OTU) IDs such as, but not limited to U.S. Pat. Nos. 4,474,380, 4,465,907, 4,327,141, 446,058 and/or 4,481,427.

In the instance of bacterial compositions provided herein, the one or more bacterial types present in the composition can be independently purified from one or more other bacteria produced and/or present in the material or environment containing the bacterial type. Bacterial compositions and the bacterial components thereof are generally purified from residual habitat products. In the instance of bacterial conditioned medium or cell pellets, these are considered pure if derived from an isolated bacteria, or combination of bacteria intentionally mixed (e.g., one or more bacteria, which when mixed, result in the production of metabolites or proteins not produced or not produced efficiently in isolation).

### BRIEF DESCRIPTION OF THE DRAWINGS

The foregoing and other objects, features and advantages will be apparent from the following description of particular embodiments of the invention, as illustrated in the accompanying drawings. The drawings are not necessarily to scale; emphasis instead being placed upon illustrating the principles of various embodiments of the invention.

FIG. 1 shows the inhibition of E. coli growth as measured in CFU/ml.

FIG. 2 shows the inhibition of E. coli growth upon co-culture with gut microbiota isolates as measured in CFU/ml. Error bars represent standard deviation.

FIG. 3 shows the absence of Enterobacteriaceae blooming sample from stool donor 8 in a gut simulator model.

FIG. 4 shows that a composition of C. bifermentans KLE 2329, V. ratti KLE 2365, G. pamelaeae, inhibits growth of E. coli. Error bars represent standard deviation.

FIGS. 5-7 show the effect of the bacterial composition containing C. bifermentans KLE 2329, V. ratti KLE 2365, G. pamelaeae in ameliorating disease in a dextran sodium sulfate mouse model of colitis. Percent survival of 8-week old C57BL/6 mice with DSS-induced colitis (3.5% DSS for 5-7 days) treated with the C. bifermentans, V. ratti, G. pamelaeae, E. coli Nissle 1917, Dialister invisus (all 10\hat{ }8 CFU/kg), or a vehicle control (Colitis control), 20% glycerol) daily for 3 days by oral gavage. Bacterial cocktail was added after 5 days of 3.5% DSS in drinking water (FIG. 5); or after 7 days of 3.5% DSS in drinking water (FIG. 6) or before administration for 7 days of 3.5% DSS for 7 days in drinking water (FIG. 7). In FIG. 7, the line connecting the data points related to Non-colitis control completely overlap with the data points related to the line connecting the data points related to treatment with the composition of C. bifermentans KLE 2329, V. ratti KLE 2365, G. pamelaeae.

FIG. 8 shows the gut microbiome composition of PTLDS subjects and healthy controls.

FIG. 9 illustrates the subclassification of PTLDS cohort into Group 1, Group 2, and Group 3.

FIG. 10 shows abundance boxplots of the five most important features that distinguish the fecal microbiome in PTLDS from the AGP healthy and ICU cohorts.

FIG. 11 represents ranked area under receiver operating characteristic curve (AUROC) reported by Duvallet et al. (2017), Nat. Commun. 8:1784, for the classification of the fecal microbiome in each disease versus a healthy control cohort. In FIG. 11, the following abbreviations indicate the following terms: ART, arthritis; ASD, autism spectrum disorder; CDI, Clostridium difficile infection; CRC, colorectal cancer; EDD, enteric diarrheal disease; HIV, human immunodeficiency virus; IBD, inflammatory bowel disease; LIV, liver disease; NASH, nonalcoholic steatohepatitis; nonCDI, non-Clostridium difficile infection; OB, obesity; PAR, Parkinson's disease; T1D, type I diabetes.

### DETAILED DESCRIPTION OF THE DISCLOSURE

### I. Introduction

The microbiome of the human intestine is a complex ecosystem consisting of several hundred, mostly anaerobic, species. To maintain colonization of the gut lumen and maximize growth in the presence of nutritional competitors, highly diverse metabolic pathways have evolved, with each microbe utilizing a different strategy for nutrient acquisition and utilization. Conditions and diseases leading to intestinal inflammation are accompanied by a severe disruption of the composition of the microbiome characterized by an expansion of facultative anaerobic Enterobacteriaceae. A disruption of this balanced community structure during episodes of disease is termed dysbiosis, which may often be characterized by the increase in prominence of bacteria that do not belong to the classes Bacteroidia or Clostridia. The most robust pattern observed during inflammation in the distal gut is an expansion of facultative anaerobic Enterobacteriaceae (class Gammaproteobacteria, phylum Proteobacteria) within the microbiome. Enterobacteriaceae normally account for only a small fraction (approximately 0.1%) of the microbiota in the large bowel, however a bloom of this family can be observed in various settings of gut inflammation.

For example, the relative luminal abundance of Enterobacteriaceae is elevated dramatically in mouse models of IBD, in which colitis is induced by a chemical trigger or by genetic predisposition. An increased prevalence of Enterobacteriaceae is also observed in patients with Crohn's disease, an IBD of unknown etiology. Antibiotic treatment raises the inflammatory tone of the intestinal mucosa, which is accompanied by a luminal bloom of Escherichia coli or Citrobacter rodentium (both members of the family Enterobacteriaceae). Similarly, repeated courses of antibiotics are associated with the development of irritable bowel syndrome in humans, a condition characterized by low-level intestinal inflammation, diarrhea, and a gut microbiota containing a heightened abundance of Proteobacteria belonging to the families Enterobacteriaceae, Pasteurellaceae, and Pseudomonadaceae. Enterobacteriaceae dominate the gut microbiota in preterm infants with necrotizing enterocolitis. The association of Lyme disease and PTLDS with levels of elevated Blautia and decreased Bacteroides have been identified by the present inventors in many PTLDS patients. These observations collectively support the utility of the microbiome profile in designing therapeutic strategies and diagnostic tools to combat disease. While therapeutic options are available for treatment of conditions and diseases associated with inflammation, the treatment outcome often is still unsatisfactory. An important consequence of the associated co-morbidities and treatment failure is reduced quality of life. Compositions described herein inhibit Enterobacteriaceae bloom, thereby facilitating therapeutic strategies for treating diseases associated with inflammation.

Among the diseases associated with inflammation and autoimmune response, Lyme disease presents a unique challenge. The etiology of Lyme disease and PTLDS is not understood, and objective diagnostic tools are lacking. Along with unknown etiopathology and a diverse range of symptoms, diagnosing PTLDS remains challenging. Although a clinical case definition proposed by the Infectious Diseases Society of America (IDSA) in 2006 has served as a specific research tool, there is no ready biological method for diagnosing PTLDS. While clinical biomarkers associated with PTLDS have been observed, efficacious diagnostic methods and therapies remain elusive.

A positron emission tomography (PET) brain imaging study among patients with PTLDS demonstrated elevated microglial activation compared to that of controls, congruent with localized inflammation. Additional research has shown that a greater B. burgdorferi-specific plasma blast response prior to treatment favors a resolution of symptoms rather than the development of PTLDS, which indicates that even before treatment, a patient's immunological landscape plays an important role in the development of PTLDS. Compared to healthy controls, patients with PTLDS have significantly elevated expression of interferon alpha, greater antibody reactivity to brain antigens, increased levels of the chemokine CCL 19 and the cytokine interleukin 23 (IL-23), and a decrease in the CD57 lymphocyte subset. Furthermore, patients have a higher risk of developing new-onset autoimmune joint diseases after a Lyme erythema migrans rash. Therefore, while the etiopathology is still unknown, these markers indicate biological abnormalities among patients with PTLDS. Since PTLDS symptoms present similarly to diseases in which the microbiome is implicated, the present inventors reasoned that the same may be true for the gut microbiome of patients with PTLDS.

### II. Compositions of the Disclosure

In some embodiments, the present disclosure provides compositions for the treatment of disease. In some embodiments, the present disclosure provides compositions for the prevention of disease. The compositions of the present disclosure may be used in the treatment of one or more diseases associated with inflammation, such as, but not limited to Lyme disease, PTLDS, IBD and/or Crohn's disease. In some embodiments, the inflammation may be gastrointestinal-associated inflammation.

Compositions of the present disclosure may include a bacterial population of one or more bacterial species. Currently, therapeutic modalities utilizing microbes and/or microbiome to treat IBD are restricted to fecal microbiota transplant (FMT). In FMT, a stool sample obtained from a healthy donor is transplanted to a recipient in need. However, FMT for IBD has been only moderately successful.

In some embodiments, the compositions of the present disclosure may include one or more commensal microbial species found in the gastrointestinal tract. In some aspects, the compositions of the present disclosure may be a defined microbial consortium. As used herein, the term “microbial consortium” refers to two or more species of microbes, e.g., bacteria, that live symbiotically. In some embodiments, the compositions of the present disclosure may be a defined microbial consortium of three bacterial species that are capable of inhibiting the growth of Enterobacteriaceae in vitro or in vivo.

In some embodiments, the compositions of the present disclosure may be used to inhibit the growth of at least one species of Enterobacteriaceae. During gastrointestinal-associated inflammation, the proinflammatory family of bacteria, Enterobacteriaceae, increase in relative abundance. This increase in Enterobacteriaceae has been associated with various diseases, for example, with IBD. In mice, it has been shown that the increase in Enterobacteriaceae may induce IBD. Preventing the increase in Enterobacteriaceae may therefore prevent IBD development. Thus, the prevention or reduction of this increase in Enterobacteriaceae is an attractive therapeutic avenue for IBD treatment. Currently, therapeutic strategies targeting Enterobacteriaceae are not available. The present disclosure describes compositions that contain a bacterial population of one or more bacterial species that inhibit the growth of one or more species of the Enterobacteriaceae family. During gastrointestinal inflammation, the immune system releases compounds which eventually react and form available nitrate, dimethyl sulfoxide (DMSO), and trimethylamine oxide (TMAO). Enterobacteriaceae are more efficient compared to commensal gut microbes at using these compounds to generate energy, which allows for the increase in Enterobacteriaceae to occur. In some embodiments, one or more of the bacterial species in the disclosed compositions may inhibit Enterobacteriaceae in vitro or in vivo by competing with Enterobacteriaceae for nutrients. The present disclosure describes compositions that include one or more bacterial species that contain the highest genome copy number, compared to other gut bacteria, of a gene which allows energy to be produced using DMSO. When such bacterial species are grown together with one or more species of Enterobacteriaceae in the presence of DMSO, the competition for DMSO leads to growth inhibition of Enterobacteriaceae. In some embodiments, the compositions include one or more bacterial species that are able to inhibit the growth of Enterobacteriaceae in vitro (or in vivo) in the presence of inflammation-associated molecule: nitrate.

Non-limiting examples of genera of Enterobacteriaceae inhibited by the compositions of the present disclosure include Escherichia, Biostraticola, Buttiauxella, Cedecea, Citrobacter, Cronobacter, Enterobacillus, Enterobacter, Franconibacter, Gibbsiella, Izhakiella, Klebsiella, Kluyvera, Kosakonia, Leclercia, Lelliottia, Limnobaculum, Mangrovibacter, Metakosakonia, Phytobacter, Pluralibacter, Pseudescherichia, Pseudocitrobacter, Raoultella, Rosenbergiella, Saccharobacter, Salmonella, Scandinavium, Shigella, Shimwellia, Siccibacter, Trabulsiella, and/or Yokenella. In one embodiment, the Enterobacteriaceae species inhibited by the compositions of the present disclosure is Escherichia coli (E. coli).

The compositions of the present disclosure may include more than 1 species of bacteria, more than 10 species of bacteria, 20 species of bacteria, 30 species of bacteria, 40 species of bacteria, 50 species of bacteria, 60 species of bacteria, 70 species of bacteria, 80 species of bacteria, 90 species of bacteria, 100 species of bacteria, 200 species of bacteria, 300 species of bacteria, 400 species of bacteria, more than 500 species of bacteria or more than 1000 species of bacteria. According to a particular embodiment, the composition ranges from 10-10,000 species of bacteria, between 100-10,000 species of bacteria or between 1000-10,000 species of bacteria.

In some embodiments, the compositions of the present disclosure include one or more bacterial species such as, but not limited to, Gordonibacter pamelaeae, Clostridium bifermentans, Veillonella ratti, Paraclostridium benzoelyticum, Sutterella wadsworthia, Alisteps onderdonkii, Barnesiella intestinihominis, Clostridium hathewayi, Bifidobacterium catenulatum, Anaerinibacillus anaerinlyticus, Coprobacillus catenformis, and/or Coprococcus comes. In some embodiments, the composition of the present disclosure includes one or more bacterial species with a 16S rDNA or 16S rRNA sequence bearing sequence identity to the 16S rRNA or 16S rDNA of a species such as but not limited to Gordonibacter pamelaeae, Clostridium bifermentans, Veillonella ratti, Paraclostridium benzoelyticum, Sutterella wadsworthia, Alisteps onderdonkii, Barnesiella intestinihominis, Clostridium hathewayi, Bifidobacterium catenulatum, Anaerinibacillus anaerinlyticus, Coprobacillus catenformis, and/or Coprococcus comes. The sequence identity percentage of the 16S rRNA of the bacterial species in the compositions may be at least 50%, at least 60%, at least 70%, at least 80%, at least 90%, at least 91%, at least 92%, at least 93%, at least 94%, at least 95%, at least 96%, at least 97%, at least 98%, and/or at least 99% to the 16S rRNA or 16S rDNA of Gordonibacter pamelaeae, Clostridium bifermentans, Veillonella ratti, Paraclostridium benzoelyticum, Sutterella wadsworthia, Alisteps onderdonkli, Barnesiella intestinihominis, Clostridium hathewayi, Bifidobacterium catenulatum, Anaerinibacillus anaerinlyticus, Coprobacillus catenformis, and/or Coprococcus comes.

In some embodiments, the compositions of the present includes one or more bacteria with a 16S rDNA sequence with at least about 97% identical to a 16S rDNA sequence of Gordonibacter pamelaeae, Clostridium bifermentans, Veillonella ratti, Paraclostridium benzoelyticum, Sutterella wadsworthia, Alisteps onderdonkii, Barnesiella intestinihominis, Clostridium hathewayi, Bifrdobacterium catenulatum, Anaerinibacillus anaerinlyticus, Coprobacillus catenformis, and Coprococcus comes.

In some embodiments, the compositions of the present disclosure includes a bacterial population of Gordonibacter pamelaeae, Veillonella ratti, and/or Clostridium bifermentans. In some embodiments, the compositions of the present disclosure includes a bacterial population of Gordonibacter pamelaeae. In some embodiments, the compositions of the present disclosure includes a bacterial population of Veillonella ratti. In some embodiments, the compositions of the present disclosure includes a bacterial population of Clostridium bifermentans.

When more than one bacterial species is present in the bacterial population of the compositions described herein, the ratio of the bacterial species present may be tuned to achieve the optimal growth inhibition of Enterobacteriaceae. In some embodiments, the ratio of one bacterial species to the other bacterial species may be 1, 2, 3, 4, 5, 6, 7, 8, 9, 10, 20, 30, 40, 50, 60, 70, 80, 90 or 100. In some embodiments, the ratio of one bacterial species to the other bacterial species may be in the range of 1-10, 5-15, 10-20, 15-25, 20-30, 25-35, 30-40, 35-45, 40-50, 45-55, 50-60, 55-65, 60-70, 65-75, 70-80, 75-85, 80-90, 85-95, 90-100. In some embodiments, two bacterial species may be present in the compositions described herein wherein the ratio of two species with respect to each other may be 1:1, 1:2, 2:1, 1:3, or 3:1. In some embodiments, three bacterial species may be present in the compositions described herein wherein the ratio of three species with respect to each other may be 1:1:1, 2:1:1, 1:2:1, 1:1:2, 2:2:1, 2:1:2, 3:1:1, 1:3:1, 1:1:3, 3:3:1, or 3:1:3.

Compositions of the disclosure may contain at least one colony-forming unit (CFU) of the bacterial populations described herein. As used herein, a CFU is defined as a single, viable propagule that produces a single colony (a population of the cells visible to the naked eye) on an appropriate semisolid growth medium. CFU may be used to denote the CFU of the entire bacterial population present in the composition or of each bacterial species present in the composition. In some embodiments the CFU of the composition and/or the CFU of each of the bacterial species of the composition may be, but is not limited to, 1×10, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, and/or 1×10²⁰. If more than one bacterial species are present in the bacterial population then each bacterial species may have a different CFU.

The growth of at least one species of Enterobacteriaceae may be inhibited by the compositions described herein. In some embodiments, the compositions of the present disclosure may inhibit the growth of Escherichia coli, a species of Enterobacteriaceae. The extent of Enterobacteriaceae growth inhibition achieved by the compositions may be 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, or a1000 fold. The extent of Enterobacteriaceae growth inhibition achieved by the compositions may be 1-10 fold, 5-15 fold, 10-20 fold, 15-25 fold, 20-30 fold, 25-35 fold, 30-40 fold, 35-45 fold, 40-50 fold, 45-55 fold, 50-60 fold, 55-65 fold, 60-70 fold, 65-75 fold, 70-80 fold, 75-85 fold, 80-90 fold, 85-95 fold, 90-100 fold, 95-10⁵ fold, 10-100 fold, or 100-1000 fold.

In some embodiments, the compositions of the present disclosure may be utilized as a probiotic. As used herein, the term “probiotic” refers to a combination of live beneficial bacteria that are found in healthy individuals of the population. In some embodiments, probiotics promote digestion, prevent the proliferation of disease causing bacteria, produce vitamins, and modulate immune responses. In some embodiments, the compositions of the present disclosure may include a bacterial population of at least one species that is non-pathogenic. In some embodiments, the compositions may include microbial competitors of non-inflammation-associated nutrients of Enterobacteriaceae.

In one embodiment, the compositions of the present disclosure may inhibit the growth Enterobacteriaceae that are antibiotic-resistant. Non-limiting examples of antibiotics to which Enterobacteriaceae may be resistant to, include erythromycin, amoxicillin and/or tetracycline.

In some embodiments, the compositions of the present disclosure may be scaled up for production. Compositions of the disclosure may also be cost-effective in production compared to FMT. In some embodiments, compositions of the disclosure may be provided to a subject in need as a traditional probiotic without hospital time nor constant medical supervision.

### III. Methods of the Disclosure

Provided herein are methods of treating and/or preventing Lyme disease, PTLDS, inflammation (e.g., IBD), and/or Enterobacteriaceae-associated dysbiosis of the human gastrointestinal tract, such as food-borne illness in a subject with the compositions described herein. The methods include contacting a subject with, or administering to a subject, one or more compositions described herein. The present inventors identified the expansion of proinflammatory Enterobacteriaceae in patients with PTLDS, suggesting that the compositions described herein, which were developed to inhibit the growth of Enterobacteriaceae, may also be useful to treat PTLDS. In some embodiments, the subject may be a human subject. The inflammation may be associated with a condition such as, but not limited to colitis, IBD and/or Crohn's disease.

Methods of treating a subject with PTLDS are described herein. A representative method involves contacting a subject with, or administering to a subject, a compositions described herein. The methods of the present disclosure may further involve administering the compositions to a subject followed by evaluating the subject for one or more symptoms associated with PTLDS. Non-limiting examples of symptoms associated with PTLDS include, arthralgias, sleep disruption, headache, neurocognitive difficulties, muscle and joint pain, fatigue and/or musculoskeletal pain. Amelioration of one or more of the symptoms associated with PTLDS is expected upon treatment with the compositions of the disclosure.

In some embodiments, the present disclosure provides methods of treating inflammation as well as methods of preventing inflammation. In some embodiments, the inflammation may be associated with a disease. Non-limiting examples of diseases associated with inflammation include IBD, colitis, allergy, asthma, autoimmune diseases, coeliac disease, glomerulonephritis, and/or hepatitis. In some aspects, the present disclosure provides methods of improving the survival of the subject. The improvement in the survival of the subject may be achieved by contacting the subject with the compositions of the disclosure. In some embodiments, the survival of the subject treated with the compositions of the disclosure may be improved by 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, or 100%. In some embodiments, the survival of the subject treated with the compositions of the disclosure may be improved by 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, 90-100%, or 95-100%. In one embodiment, the survival of the subject treated with the compositions of the disclosure may be improved by from about 80% to about 100%.

In some embodiments, the methods of the present disclosure may include methods of diagnosing Lyme disease or PTLDS in a subject. Diagnosis of PTLDS may be derived from the microbiome signature encountered in PTLDS. Microbiome signatures associated with PTLDS are described herein and in Morrissette et al. (2020), mBio 11:e02310-20 (the contents of which are herein incorporated by reference in their entirety). In some embodiments, microbiome signatures may be relied upon as proxy for microbiome composition and/or activity. As used herein, the term “microbiome signature” includes data points that are indicators of microbiome composition and/or activity. Accordingly, changes in microbiomes can be detected and/or analyzed through detection of one or more features of microbiome signatures. In some embodiments, a microbiome signature includes information relating to absolute amount of one or more types of bacterial species, and/or products thereof. In some embodiments, a microbiome signature includes information relating to relative amounts of five, ten, twenty or more types of bacterial species and/or products thereof.

Methods of diagnosing PTLDS in a subject may involve obtaining a sample from the subject. The sample may be a stool sample, a blood sample, an oral swab, an anal swab, and/or a hair sample. For analysis of the microbiome signature, 16S rRNA is extracted from the sample using methods known in the art. In some embodiments, an additional step is performed wherein the 16S rRNA is reverse transcribed to 16S rDNA. The 16S rDNA or 16S rRNA may then be utilized to prepare the microbiome signature of the sample. In some embodiments, microbiome signature may be prepared by gene sequencing the 16S rDNA. In some embodiments, the microbiome signature may be prepared by utilizing polymerase chain reactions to amplify a cohort of bacterial genera contained in the microbiome signature. In some embodiments, the microbiome signature comprises bacterial genera such as, but not limited to, Blautia, Clostridium, Roseburia, Staphylococcus, Bacteroides Parabacteroides, Barnesiella, Faecalibacterium, Enterococcus, Escherichia, Akkermansia, Alistipes, Barnesiella, Bfidobacterium, Catenibacterium, Collinsella, Coprococcus, Dialister, Dorea, Eubacterium, Lactobacillus, Methanobrevibacter, Prevotella, Ruminococcus, Shigella, Streptococcus, and Subdoligranulum.

In some embodiments, a microbiome signature includes information relating to presence, level, and/or activity of at least one of bacterial species. In some embodiments, a microbiome signature includes information relating to the presence, level, and/or activity of between 3 and 100 types of bacterial species. In some embodiments, a microbiome signature includes information relating to presence, level, and/or activity of between 100 and 1000 or more types of bacterial species. In some embodiments, a microbiome signature includes information relating to presence, level, and/or activity of substantially all types of bacterial species within the microbiome. In some embodiments, a microbiome signature comprises a level or set of levels of one, five, or ten or more types of bacterial species or components or products thereof. In some embodiments, a microbiome signature comprises a level or set of levels of five or ten or more DNA sequences. In some embodiments, a microbiome signature comprises a level or set of levels of ten or more 16S rRNA gene sequences. In some embodiments, a microbiome signature comprises a level or set of levels of 18S rRNA gene sequences. In some embodiments, a microbiome signature comprises a level or set of levels of five or ten or more RNA transcripts. In some embodiments, a microbiome signature comprises a level or set of levels of five or ten or more proteins. In some embodiments, a microbiome signature comprises a level or set of levels of five or ten or more metabolites.

In order to classify a microbe as belonging to a particular genus, it may include at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the particular genus. According to a particular embodiment, the sequence homology is at least 95%. According to another embodiment, in order to classify a microbe as belonging to a particular species, it must comprise at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the particular species. According to a particular embodiment, the sequence homology may be at least 97%.

In some embodiments, the bacterial genera in the microbiome signature may be identified as follows. Sequences derived from 16S rDNA or 16SrDNA gene sequencing studies may be clustered into bins called ‘Operational Taxonomic Units” (OTUs) based upon similarity. The similarity between a pair of sequences is computed as the percentage of sites that agree in a pairwise sequence alignment. In some embodiments, the common 16S rRNA sequence similarity threshold may be 97%. In some embodiments, the microbiome signature may include bacterial genera with OTU IDs such as, but not limited to U.S. Pat. Nos. 4,474,380, 4,465,907, 4,327,141, 446058 and/or 4481427.

The bacterial genera included in microbiome signature may have a relative abundance level of from about 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, or 100%. In some embodiments, the relative abundance of the bacterial genera may be 0.001%-00.1%, 0.1%-0.2%, 1-10%, 5-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, 90-100/o, or 95-100%. As anon-limiting example, the relative abundance may be 8.86%. In some embodiments, the relative abundance may be 0.0024%. In some embodiments, the relative abundance may be 0.15%.

### IV. Pharmaceutical and Alternative Compositions

The microbial composition may be formulated as a pharmaceutical composition. As used herein, the term “pharmaceutical composition” refers to a preparation of one or more of the active ingredients described herein with other chemical components such as physiologically suitable carriers and excipients. In some embodiments, the purpose of a pharmaceutical composition is to facilitate administration of the composition to an organism. As used herein, the term “active ingredient” refers to one or more bacterial species or bacterial population and/or compositions of the present disclosure accountable for the biological effect. As used herein, the term “physiologically acceptable carrier” may be interchangeably used refer to a carrier or a diluent that does not cause significant irritation to an organism and does not abrogate the biological activity and properties of the administered composition. The physiologically acceptable carrier is selected such that the bacterial species within the composition remain viable.

As used herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present disclosure may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

Pharmaceutical compositions suitable for use in context of the present disclosure include compositions wherein the active ingredients are contained in an amount effective to achieve the intended purpose. More specifically, a therapeutically effective amount means an amount of active ingredients (e.g., a bacterial population of one or more bacterial species) effective to prevent, alleviate or ameliorate symptoms of a disorder (e.g., PTLDS) or prolong the survival of the animal being treated. In general, a pharmaceutical composition may be delivered as a drug, a pharmaceutical preparation, probiotic, prebiotic, a capsule, a tablet, a caplet, a pill, a troche, a lozenge, a powder, a granule, or in any other suitable form.

Alternatively, the microbial composition (and its constituents) can be delivered as a dietary ingredient, a food, a food supplement, a medical food, or a combination thereof.

### V. Administration and Dosing

In some embodiments, compositions or the pharmaceutical compositions of the present disclosure may be administered via one or more administration routes. In various embodiments, administration may be oral, enteral (into the intestine), transdermal, intravenous bolus, intralesional (within or introduced directly to a localized lesion), intrapulmonary (within the lungs or its bronchi), diagnostic, intraocular (within the eye), transtympanic (across or through the tympanic cavity), intravesical infusion, sublingual, nasogastric (through the nose and into the stomach), spinal, intracartilaginous (within a cartilage), insufflation (snorting), rectal, intravascular (within a vessel or vessels), buccal (directed toward the cheek), dental (to a tooth or teeth), intratesticular (within the testicle), intratympanic (within the aurus media), percutaneous, intrathoracic (within the thorax), submucosal, cutaneous, epicutaneous (application onto the skin), dental intracomal, intramedullary (within the marrow cavity of a bone), intra-abdominal, epidural (into the dura matter), intramuscular (into a muscle), intralymphatic (within the lymph), iontophoresis (by means of electric current where ions of soluble salts migrate into the tissues of the body), subcutaneous (under the skin), intragastric (within the stomach), nasal administration (through the nose), transvaginal, intravenous drip, endosinusial, intraprostatic (within the prostate gland), soft tissue, intradural (within or beneath the dura), subconjunctival, oral (by way of the mouth), peridural, parenteral, intraduodenal (within the duodenum), intracistemal (within the cistema magna cerebellomedularis), periodontal, periarticular, biliary perfusion, intracoronary (within the coronary arteries), intrathecal (within the cerebrospinal fluid at any level of the cerebrospinal axis), intrameningeal (within the meninges), intracavemous injection (into a pathologic cavity) intracavitary (into the base of the penis), intrabiliary, subarachnoid, intrabursal, ureteral (to the ureter), intratendinous (within a tendon), auricular (in or by way of the ear), intracardiac (into the heart), enema, intraepidermal (to the epidermis), intraventricular (within a ventricle), intramyocardial (within the myocardium), intratubular (within the tubules of an organ), vaginal, sublabial, intracorporus cavemosum (within the dilatable spaces of the corporus cavemosa of the penis), intradermal (into the skin itself), intravitreal (through the eye), perineural, cardiac perfusion, irrigation (to bathe or flush open wounds or body cavities), in ear drops, endotracheal, intraosseous infusion (into the bone marrow), caudal block, intrauterine, transtracheal (through the wall of the trachea), intra-articular, intracomeal (within the cornea), endocervical, extracorporeal, intraspinal (within the vertebral column), transmucosal (diffusion through a mucous membrane), topical, photopheresis, oropharyngeal (directly to the mouth and pharynx), occlusive dressing technique (topical route administration which is then covered by a dressing which occludes the area), transplacental (through or across the placenta), intrapericardial (within the pericardium), intraarterial (into an artery), interstitial, intracerebral (into the cerebrum), intracerebroventricular (into the cerebral ventricles), intrapleural (within the pleura), infiltration, intrabronchial, intrasinal (within the nasal or periorbital sinuses), intraductal (within a duct of a gland), intracaudal (within the cauda equine), nerve block, retrobulbar (behind the pons or behind the eyeball), intravenous (into a vein), intra-amniotic, conjunctival, intrasynovial (within the synovial cavity of a joint), gastroenteral, intraluminal (within a lumen of a tube), electro-osmosis, intraileal (within the distal portion of the small intestine), intraesophageal (to the esophagus), extra-amniotic administration, hemodialysis, intragingival (within the gingivae), intratumor (within a tumor), eye drops (onto the conjunctiva), laryngeal (directly upon the larynx), urethral (to the urethra), intravaginal administration, intraperitoneal (infusion or injection into the peritoneum), respiratory (within the respiratory tract by inhaling orally or nasally for local or systemic effect), intradiscal (within a disc), ophthalmic (to the external eye), and/or intraovarian (within the ovary).

In some embodiments, pharmaceutical compositions may be administered by intraarticular administration, extracorporeal administration, intrabronchial administration, endocervical administration, endosinusial administration, endotracheal administration, enteral administration, epidural administration, intra-abdominal administration, intrabiliary administration, intrabursal administration, oropharyngeal administration, interstitial administration, intracardiac administration, intracartilaginous administration, intracaudal administration, intracavemous administration, intracerebral administration, intracorporous cavemosum, intracavitary administration, intracomeal administration, intracistemal administration, cranial administration, intracranial administration, intradermal administration, intralesional administration, intratympanic administration, intragingival administration, intraocular administration, intradiscal administration, intraductal administration, intraduodenal administration, ophthalmic administration, intradural administration, intraepidermal administration, intraesophageal administration, nasogastric administration, nasal administration, laryngeal administration, intraventricular administration, intragastric administration, intrahepatic administration, intraluminal administration, intravitreal administration, intravesicular administration, intralymphatic administration, intramammary administration, intramedullary administration, intrasinal administration, intrameningeal administration, intranodal administration, intraovarian administration, intraperitoneal administration, intrapleural administration, intraprostatic administration, intraluminal administration, intraspinal administration, intrasynovial administration, intratendinous administration, intratesticular administration, subconjunctival administration, intracerebroventricular administration, epicutaneous administration, intravenous administration, retrobulbar administration, periarticular administration, intrathoracic administration, subarachnoid administration, intratubular administration, periodontal administration, transtympanic administration, transtracheal administration, intratumor administration, vaginal administration, urethral administration, intrauterine administration, oral administration, gastroenteral administration, parenteral administration, sublingual administration, ureteral administration, percutaneous administration, peridural administration, transmucosal administration, perineural administration, transdermal administration, rectal administration, soft tissue administration, intraarterial administration, subcutaneous administration, topical administration, extra-amniotic administration, ear drops, or intravesical infusion.

Compositions of the present disclosure may be administered orally but any suitable route of administration may be employed for providing a subject with an effective dosage of drugs of the chemical compositions described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In certain embodiments, it may be advantageous that the compositions described herein be administered orally.

Compositions of the present disclosure may be administered in the conventional manner by any route where they are active. Administration can be systemic, parenteral, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration of the composition of the present disclosure (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

A metered dose of the composition can be provided from a reservoir of the composition. In addition, predetermined dosages can be provided, for example, suppository forms can be provided for insertion into the nose or rectum having a predetermined dosage. Kits can be provided, where prepared dosage forms and instructions for administering the dosages are included.

Dosage amounts and intervals of the compositions of the present disclosure may be adjusted individually to provide microbe numbers sufficient to induce an effect (such as, but not limited to, minimal effective concentration or MEC). The MEC will vary for each preparation, but can be estimated from in vitro data. Dosages necessary to achieve the MEC will depend on individual characteristics and route of administration. The amount of a composition to be administered will, of course, be dependent on the animal being treated (e.g., age, weight) and the manner of administration.

In some embodiments, compositions of the present disclosure are provided in one or more doses and are administered one or more times to subjects. Some compositions are provided in only a single administration. Some pharmaceutical formulations are provided according to a dosing schedule that includes two or more administrations. Each administration may be at the same dose or may be different from a previous and/or subsequent dose. In some embodiments, subjects are provided an initial dose that is higher than subsequent doses (referred to herein as a “loading dose”). In some embodiments, doses are decreased over the course of administration. Dosing schedules may include compositions administration from about every 2 hours to about every 10 hours, from about every 4 hours to about every 20 hours, from about every 6 hours to about every 30 hours, from about every 8 hours to about every 40 hours, from about every 10 hours to about every 50 hours, from about every 12 hours to about every 60 hours, from about every 14 hours to about every 70 hours, from about every 16 hours to about every 80 hours, from about every 18 hours to about every 90 hours, from about every 20 hours to about every 100 hours, from about every 22 hours to about every 120 hours, from about every 24 hours to about every 132 hours, from about every 30 hours to about every 144 hours, from about every 36 hours to about every 156 hours, from about every 48 hours to about every 168 hours, from about every 2 days to about every 10 days, from about every 4 days to about every 15 days, from about every 6 days to about every 20 days, from about every 8 days to about every 25 days, from about every 10 days to about every 30 days, from about every 12 days to about every 35 days, from about every 14 days to about every 40 days, from about every 16 days to about every 45 days, from about every 18 days to about every 50 days, from about every 20 days to about every 55 days, from about every 22 days to about every 60 days, from about every 24 days to about every 65 days, from about every 30 days to about every 70 days, from about every 2 weeks to about every 8 weeks, from about every 3 weeks to about every 12 weeks, from about every 4 weeks to about every 16 weeks, from about every 5 weeks to about every 20 weeks, from about every 6 weeks to about every 24 weeks, from about every 7 weeks to about every 28 weeks, from about every 8 weeks to about every 32 weeks, from about every 9 weeks to about every 36 weeks, from about every 10 weeks to about every 40 weeks, from about every 11 weeks to about every 44 weeks, from about every 12 weeks to about every 48 weeks, from about every 14 weeks to about every 52 weeks, from about every 16 weeks to about every 56 weeks, from about every 20 weeks to about every 60 weeks, from about every 2 months to about every 6 months, from about every 3 months to about every 12 months, from about every 4 months to about every 18 months, from about every 5 months to about every 24 months, from about every 6 months to about every 30 months, from about every 7 months to about every 36 months, from about every 8 months to about every 42 months, from about every 9 months to about every 48 months, from about every 10 months to about every 54 months, from about every 11 months to about every 60 months, from about every 12 months to about every 66 months, from about 2 years to about 5 years, from about 3 years to about 10 years, from about 4 years to about 15 years, from about 5 years to about 20 years, from about 6 years to about 25 years, from about 7 years to about 30 years, from about 8 years to about 35 years, from about 9 years to about 40 years, from about 10 years to about 45 years, from about 15 years to about 50 years, or more than every 50 years.

The desired dosage may be delivered for a duration of about 5 days to 365 days, about 5 days to 300 days, about 5 days to 300 days, about 5 days to 250 days, about 5 days to 200 days, about 5 days to 100 days, about 5 days to 60 days, about days to 30 days, about 5 days to 14 days, or about 3 days to 7 days, preferably about 21 days to 28 days.

In some embodiments, the compositions of the present disclosure may be provided at a dose of 1×10 CFU/kg, 1×10² CFU/kg, 1×10³ CFU/kg, 1×10⁴ CFU/kg, 1×10⁵ CFU/kg, 1×10⁶ CFU/kg, 1×10⁷ CFU/kg, 1×10⁸ CFU/kg, 1×10⁹ CFU/kg, 1×10¹⁰ CFU/kg, 1×10¹¹ CFU/kg, 1×10¹² CFU/kg, 1×10¹³ CFU/kg, 1× 10¹⁴ CFU/kg, 1×10¹⁵ CFU/kg, 1×10¹⁶, 1×10¹⁷ CFU/kg, 1×10¹⁸ CFU/kg, 1×10¹⁹ CFU/kg, and/or 1×10²⁰ CFU/kg.

### VI. Definitions

Administering: The term “administering” means to administer, e.g., a therapeutic agent to a patient, whereby the therapeutic positively affects the tissue or the organ to which it is targeted. The compositions described herein can be administered either alone or in combination (concurrently or serially) with other pharmaceuticals. For example, compositions can be administered in combination with other vaccines, antibiotics, antiviral agents, anti-cancer or anti-neoplastic agents, or in combination with other treatment modalities such as herbal therapy, acupuncture, naturopathy, etc.

Colony-Forming Unit: A CFU is a single, viable propagule that produces a single colony (a population of the cells visible to the naked eye) on an appropriate semisolid growth medium.

Commensalism: As used herein, the term “commensalism” refers to a long-term biological interaction in which members of one species gain benefits while those of the other species neither benefit nor are harmed. The species involved in the biological interaction are referred to as commensals.

Consortium: As used herein, the term “consortium” means a group of different species of microorganisms that act together as a community and/or are associated symbiotically.

Effective Amount: The term “effective amount” as used herein generally refers to a sufficient amount of the therapeutic agent to decrease, prevent or inhibit the disease. The amount will vary for each compound and upon known factors related to the item or use to which the therapeutic agent is applied.

Immune response: The term “immune response” as used herein refers to activity of the cells of the immune system upon exposure to a stimulus such as, but not limited to, an antigen. In one embodiment, the antigen may be derived from Borrelia species.

Modulation: The term “modulation” is art-recognized and refers to up-regulation (i.e., activation or stimulation), down-regulation (i.e., inhibition or suppression) of a response, or the two in combination or apart.

Microbiome: As used herein, the term “microbiome” refers to the totality of microbes (bacteria, fungi, protists) and their genetic elements (genomes) in a defined environment in an organism. In one embodiment, the defined environment may be the gastrointestinal tract and the microbiome associated with the gastrointestinal tract is herein referred to as the gut microbiome.

Microbial Consortium: As used herein, the term “microbial consortium” refers to two or more species of microbes, e.g., bacteria, that live symbiotically in an environment within the host.

Pharmaceutically acceptable: The term “pharmaceutically acceptable” refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier” refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

Subject: A “subject” may include a human subject for medical purposes, such as for the treatment of an existing disease, disorder, condition or the prophylactic for preventing the onset of a disease, disorder, or condition; or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

Treatment or Treating: The term “treatment” or “treating” refers to an intervention performed with the intention of preventing the development or altering the pathology or symptoms of a disorder. Accordingly, “treatment” can refer to therapeutic treatment or prophylactic or preventative measures. In some embodiments, the treatment is for therapeutic treatment. In some embodiments, the treatment is for prophylactic or preventative treatment. Those in need of treatment can include those already with the disorder as well as those in which the disorder is to be prevented. In some embodiments, the treatment is for experimental treatment.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### EXAMPLES

### Example 1. Identification of Bacteria that Inhibit Enterobacteriaceae Growth

During gastrointestinal inflammation, the immune system releases compounds which eventually react and form available nitrate, dimethyl sulfoxide (DMSO), and trimethylamine oxide (TMAO). Enterobacteriaceae (e.g., E. coli) have an advantage over other gut microbes during inflammation due to their ability to utilize DMSO), nitrate and TMAO as electron acceptors in anaerobic respiration. This results in an increase in Enterobacteriaceae in the gut during inflammation. The inventors tested if the introduction of bacteria with reductases capable of utilizing DMSO and TMAO, could be used to overcome the advantage experienced by Entereobacteriaceae and prevent their bloom. Most gut commensals lack DMSO/nitrate/TMAO reductases. The genome of Gordonibacter pamelaeae DMSZ 19378, however, includes 12 copies of the DMSO reductase gene in its genome, which is the most of any gut species. To test if G. pamelaeae could prevent an Enterobacteriaceae bloom in the presence of DMSO conditions, competition assays were performed between E. coli and G. pamelaeae with 1% DMSO under anaerobic conditions. E. coli bacteria were grown in competition with G. pamelaeae in Wilkins-Chalgren media with 0.25× glucose, 1% asparagine, and 1% DMSO. Stationary phase cells of E. coli and G. pamelaeae were combined in a 1:2 ratio and E. coli CFU/mL were determined by selective plating on MacConkey agar at time 0, 8, and 24 hr.

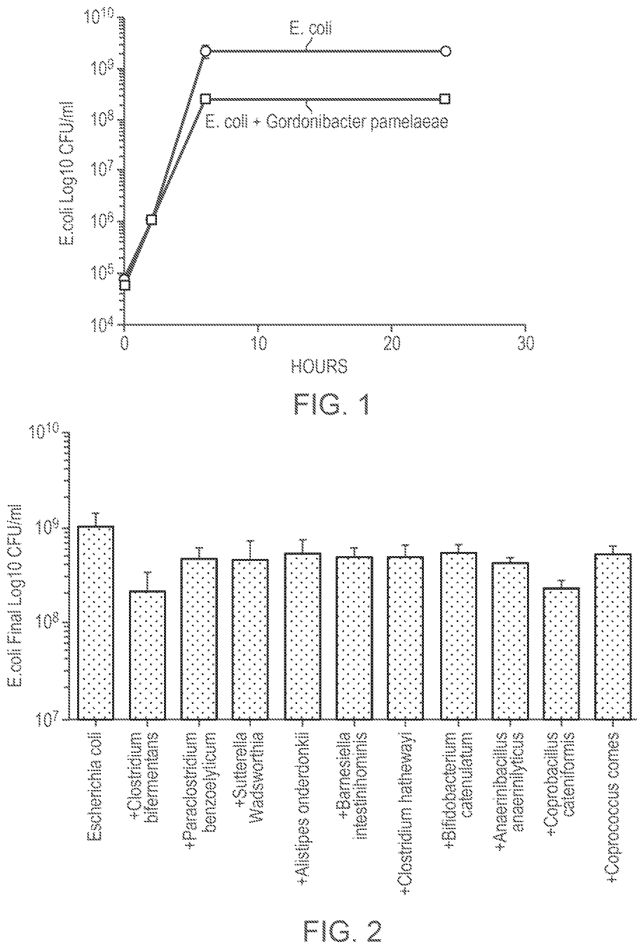

In nutrient rich media (Brain Heart Infusion or GIFU anaerobic growth medium), the growth of E. coli was not inhibited by the presence of G. pamelaeae. This is likely due to the multiple nutrients available in the nutrient rich media. In contrast, E. coli growth was inhibited by almost one-log (or 10 fold) in the Wilkins-Chalgren defined medium created for anaerobes with reduced glucose and asparagine (FIG. 1) (p=0.0006; Student's T Test). These results suggest that bacteria such as G. pamelaeae, which utilize DMSO/nitrate/TMAO, can prevent Enterobacteriaceae bloom under in vitro inflammatory conditions. Table 1 shows the inhibition of E. coli growth as measured by colony-forming unit (CFU)/ml.

TABLE 1
Growth of E. coli in the presence of G. pamelaeae

| Time | E. coli | E. coli with G. pamelaeae |
|---|---|---|
| (hours) | (CFU/mL +/− SD) | (CFU/mL +/− SD) |
| 0 | 7.33E+04 +/− 4.71E+03 | 5.50E+04 +/− 1.61E+04 |
| 2 | 1.15E+06 +/− 5.00E+04 | 1.05E+06 +/− 1.98E+05 |
| 7 | 2.20E+09 +/− 7.12E+08 | 2.40E+08 +/− 4.51E+07 |

To identify gut bacteria that inhibit the growth of E. coli in nutrient rich media such as Brain Heart Infusion media, competition assays were performed using approximately 300 gut bacterial species isolated from human stool and E. coli. The bacteria used in the assay constitutively express Light, Oxygen, or Voltage sensing (LOV) protein. The inducible LOV protein or iLOV is photo reversible and does not require oxygen to properly fold, making it ideal for using in high throughput anaerobic screening. The screen identified 15 isolates whose presence inhibited E. coli growth based on decreased fluorescence measured. The ability of ten of the fifteen isolates identified to inhibit E. coli growth was confirmed using E. coli MG1655 in Brain Heart Infusion (BHI) with 30 mM nitrate. E. col MG1655 was added in a 1:2 cell ratio with each of the isolates in Brain Heart Infusion with 30 mM nitrate. After 24 hr, the co-cultures were plated on Enterobacteriaceae selective MacConkey agar and bacterial growth was measured as CFU/ml. Each isolate reduced the final CFU/mL of E. coli when compared to the control. Notably, Clostridium bifermentans KLE 2329 yielded the greatest inhibition of E. coli MG1655, by almost one log (or approximately 5 fold). FIG. 2 and Table 2 show the inhibition of E. coli growth upon co-culture with gut microbiota isolates as measured by colony-forming unit (CFU)/ml. Error bars represent standard deviation.

TABLE 2
Growth of E.coli in the presence of other bacterial species

| Species cultured | Mean CFU/mL | SD |
|---|---|---|
| Escherichia coli | 9.89E+08 | 5.22E+08 |
| E. coli + Clostridium bifermentans | 2.12E+08 | 1.32E+08 |
| E. coli + Paraclostridium benzoelyticum | 4.56E+08 | 1.64E+08 |
| E. coli + Sutterella Wadsworthia | 4.3E+08 | 3.08E+08 |
| E. coli + Alistipes onderdonkii | 5.33E+08 | 2.29E+08 |
| E. coli + Barnesiella intestinihominis | 4.67E+08 | 1.63E+08 |
| E. coli + Clostridium hathewayi | 4.67E+08 | 2.29E+08 |
| E. coli + Bifidobacterium catenulatum | 5.33E+08 | 1.25E+08 |
| E. coli + Anaerinibacillus anaerinilyticus | 4.25E+08 | 4.71E+07 |
| E. coli + Coprobacillus cateniformis | 2.33E+08 | 4.71E+07 |
| E. coli + Coprococcus comes | 5.13E+08 | 1.36E+08 |

### Example 2. In Vitro Gut Simulator Model Studies

The Lewis Gut Simulator model (LEGS), an in vitro gut simulator model simplified from the Simulator of the Human Intestinal Microbial Ecosystem was used as previously described (O'Connor et al. (2019), PLoS ONE 14(11): e0224836, https://doi.org/10.1371/journal.pone.0224836, the entire contents of which are incorporated by reference). Briefly, the LEGS is a system that pumps diluted GIFU anaerobic growth media through a series of silicone tubing via peristaltic pump into vessels with an excretion port. Each vessel is inoculated with a stool sample diluted 10′ and fresh media is pumped at 0.101 ml/minute so that 145.44 ml of the 150 ml total volume is replaced every 24 hours. The LEGS was used as a representative of the human colon microbiota and was used to mimic Enterobacteriaceae bloom.

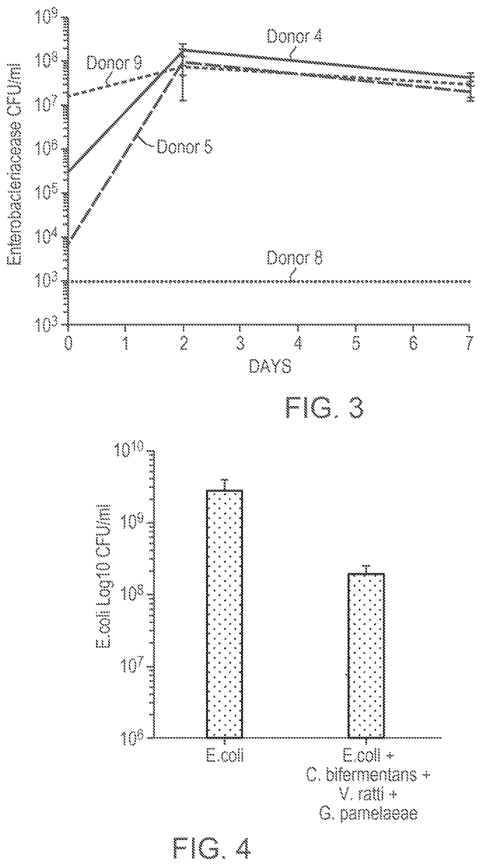

Stools from donors 4, 5, 8, and 9 were added respectively to a gut simulator model. The Enterobacteriaceae CFU/mL was calculated at day 0, 2, and 7 by plating on MacConkey agar. One of the stool samples used in the study (donor 8) was identified as being resistant to the simulator-induced Enterobacteriaceae blooms. Stool from donors 4, 5, and 9 all started with differing levels of Enterobacteriaceae, but by day 2, the levels of Enterobacteriaceae reached approximately the same CFU/mL (FIG. 3). However, stool from donor 8, which has cultivable Enterobacteriaceae at day 0, did not show an Enterobacteriaceae bloom (FIG. 3). Several other genera in sample 8 bloomed in the gut simulator. Most prominently, the genus Veillonella, which was highly represented by Veillonella ratti was observed in this sample. Veillonella was present at low abundance in samples 4, 5, and 9. These results suggested that V. ratti could inhibit Enterobacteriaceae in an environment favoring Enterobacteriaceae. Thus, V. ratti KLE 2365 was isolated for use in further studies. FIG. 3 and Table 3 show the absence of Enterobacteriaceae blooming sample from stool donor 8 in a gut simulator model.

TABLE 3
Growth of Enterobacteriaceae in the presence of donor stool samples

| | Sample Number | | | | | | | |
|---|---|---|---|---|---|---|---|---|
| | 4 | | 5 | | 9 | | 8 | |
| Day | Mean | SD | Mean | SD | Mean | SD | Mean | SD |
| 0 | 2.70E+05 | 4.24E+04 | 6.50E+03 | 3.25E+03 | 1.53E+07 | 1.02E+07 | 1.00E+03 | 0.00E+00 |
| 2 | 1.88E+08 | 6.08E+07 | 9.90E+07 | 8.67E+07 | 7.75E+07 | 3.23E+07 | 1.00E+03 | 0.00E+00 |
| 7 | 4.32E+07 | 1.15E+07 | 2.03E+07 | 8.36E+06 | 2.91E+07 | 1.46E+07 | 1.00E+03 | 0.00E+00 |

### Example 3. Inhibition of E. coli Growth by Compositions of Bacteria

C. bifermentans KLE 2329, V. ratti KLE 2365, G. pamelaeae, and E. coli MG1655 were grown in a 2:1:1:1 ratio in 1% DMSO and 30 mM nitrate in buffered (MOPS, pH 7.0) Brain Heart Infusion under anaerobic conditions. After 24 hr, the E. coli CFU/mL was calculated by plating on MacConkey agar. The bacterial composition of C. bifermentans KLE 2329, V. ratti KLE 2365, and G. pamelaeae inhibited E. coli MG1655 growth by over one log (or 14 fold) in 1% DMSO and 30 mM nitrate in buffered (MOPS, pH 7.0) BHI in vitro (FIG. 4 and Table 4).

TABLE 4
Growth of Enterobacteriaceae

| | E. coli CFU/mL +/− SD |
|---|---|
| E. coli | 1.84E+09 +/− 3.68E+08 |
| E. coli + C. | 1.29E+08 +/− 4.55E+07 |
| bifermentans, V. | |
| ratti, G. pamelaeae | |

### Example 4. Efficacy of Compositions in a Mouse Model of Colitis

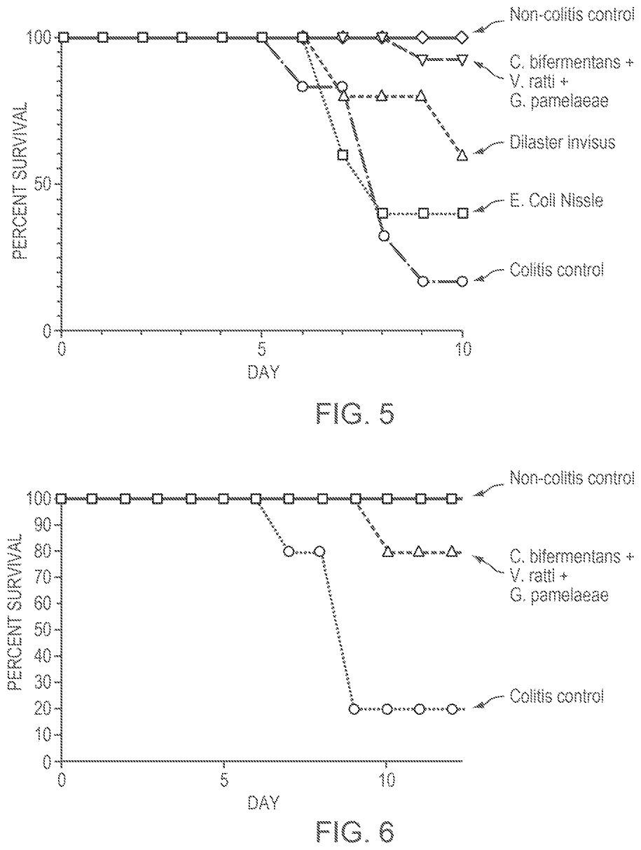

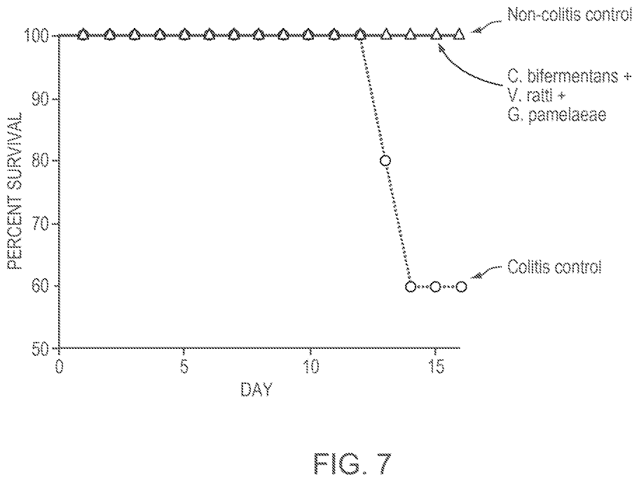

The efficacy of the bacterial cocktail containing C. bifermentans KLE 2329, V. ratti KLE 2365, G. pamelaeae was tested in a dextran sodium sulfate (DSS) mouse model of colitis at a ratio of 1:1:1. The DSS mouse model of colitis is a widely used model of IBD more closely resembling ulcerative colitis. DSS induces damage to the intestinal monolayer, causing an inflammatory response. To test the therapeutic potential of the compositions developed by the inventors, eight-week old C57/BL6 mice were given 3.5% DSS in drinking water for five to seven days to induce colitis and then given either the cocktail (10⁸ CFU), vehicle (20% glycerol), or live bacterial controls: Dialister invisus or E. coli Nissle (10\hat{ }8 CFU), daily for three days via oral gavage. To test the prophylactic potential, mice were given the bacterial cocktail or vehicle daily for three days via oral gavage, rested for 2 days, and then given 3.5% DSS in drinking water for seven days. Animals receiving the bacterial cocktail had improved survival at the end of each experiment (range 80-100% survival) compared to animals who received the vehicle control or live bacterial controls (range 16-60% survival) (FIG. 5, Table 5, FIG. 6 and Table 6). The consortium used prophylactically completely protected mice from death (FIG. 7 and Table 7). These results show that the consortium improves survival in a mouse model of colitis.

TABLE 5
Percent survival in different cohorts

| | | | C. | | |
|---|---|---|---|---|---|
| | | E. coli | bifermentans, | | |
| | Colitis | Nissle | V. ratti, G. | Non-colitis | Dialister |
| Day | control | 1917 | pamelaeae | control | invisus |
| 0 | 100 | 100 | 100 | 100 | 100 |
| 1 | 100 | 100 | 100 | 100 | 100 |
| 2 | 100 | 100 | 100 | 100 | 100 |
| 3 | 100 | 100 | 100 | 100 | 100 |
| 4 | 100 | 100 | 100 | 100 | 100 |
| 5 | 100 | 100 | 100 | 100 | 100 |
| 6 | 83.33 | 100 | 100 | 100 | 100 |
| 7 | 83.33 | 60 | 100 | 100 | 80 |
| 8 | 33.33 | 40 | 100 | 100 | 80 |
| 9 | 17 | 40 | 92.3 | 100 | 80 |
| 10 | 17 | 40 | 92.3 | 100 | 60 |

TABLE 6
Percent survival in different cohorts

| | | | C. |
|---|---|---|---|
| | | | bifermentans, |
| | Non-colitis | Colitis | V. ratti, G. |
| Day | control | control | pamelaeae |
| 0 | 100 | 100 | 100 |
| 1 | 100 | 100 | 100 |
| 2 | 100 | 100 | 100 |
| 3 | 100 | 100 | 100 |
| 4 | 100 | 100 | 100 |
| 5 | 100 | 100 | 100 |
| 6 | 100 | 100 | 100 |
| 7 | 100 | 80 | 100 |
| 8 | 100 | 80 | 100 |
| 9 | 100 | 20 | 100 |
| 10 | 100 | 20 | 80 |
| 11 | 100 | 20 | 80 |
| 12 | 100 | 20 | 80 |

TABLE 7
Percent survival in different cohorts

| | Non- | | C. bifermentans, |
|---|---|---|---|
| | colitis | Colitis | V. ratti, G. |
| Day | control | control | pamelaeae |
| 0 | 100 | 100 | 100 |
| 1 | 100 | 100 | 100 |
| 2 | 100 | 100 | 100 |
| 3 | 100 | 100 | 100 |
| 4 | 100 | 100 | 100 |
| 5 | 100 | 100 | 100 |
| 6 | 100 | 100 | 100 |
| 7 | 100 | 100 | 100 |
| 8 | 100 | 100 | 100 |
| 9 | 100 | 100 | 100 |
| 10 | 100 | 100 | 100 |
| 11 | 100 | 100 | 100 |
| 12 | 100 | 100 | 100 |
| 13 | 100 | 80 | 100 |
| 14 | 100 | 60 | 100 |
| 15 | 100 | 60 | 100 |
| 16 | 100 | 60 | 100 |

In a another study, mice treated with DSS were then treated with one dose per day for two days of 10¹ cells/kg of the composition (n=4) and given a 3-day recovery period with no intervention. Colitis control mice were given the same DSS dosage, but did not receive the composition (colitis control, n=6); non-colitis control mice did not receive DSS nor the consortium (non-colitis, n=3). The non-colitis and consortium groups had 100% survival at day 10 compared to the colitis control survival of 16% by day 10. This increase in survival rate was coupled with amelioration of colon pathology in the group treated with the C. bifermentans. V. ratti, G. pamelaeae composition. The mice treated with the composition of C. bifermentans. V. ratti, G. pamelaeae had lower colon length/weight ratios compared to the colitis control group and their colons appeared more phenotypically similar to the non-colitis controls with less blanching and the presence of stool pellets as compared to the colitis control group. These results suggest that the composition of C. bifermentans. V. ratti, G. pamelaeae improves survival and colon integrity in a mouse model of colitis.

### Example 5. Analysis of Gut Microbiome of Patients with PTLDS

Clinically, PTLDS presents similarly to an autoimmune disease. Given, the strong correlation between the immune system and the gut microbiome composition, the gut microbiome of PTLDS patients was analyzed.

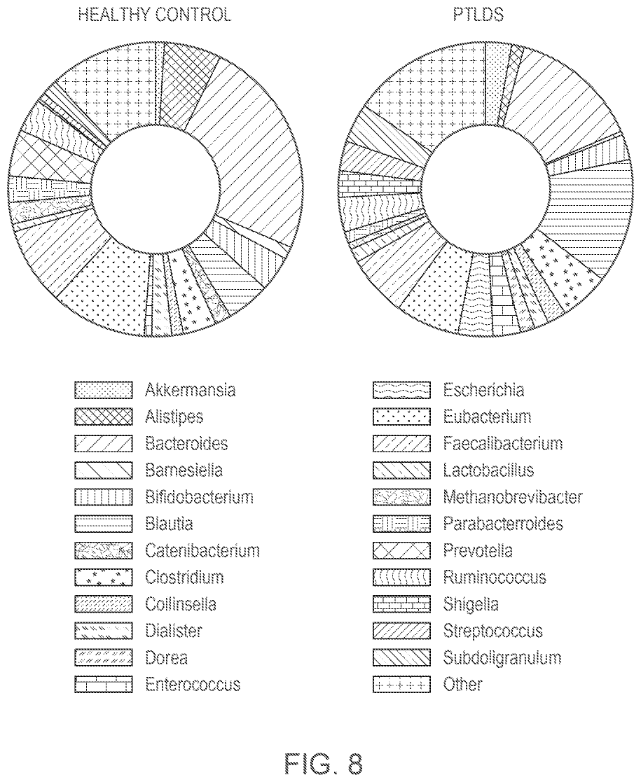

The gut microbiome of subjects with PTLDS from the John Hopkins University Lyme disease research center's Study of Lyme Immunology and Clinical Endpoints (SLICE) cohorts was analyzed. The SLICE cohort consists of a patient group with well-defined PTLDS. 16s rRNA gene sequencing was performed on stool samples from the SLICE cohort using Illumina (n=51) and Ion Torrent (n=90) technology and at Mr. DNA respectively. To analyze the 16s rRNA gene sequencing performed by Ion Torrent technology, a healthy control cohort collected at Northeastern University (n=20) (Mr. DNA) was used. The average composition based on the relative abundance of genera in the gut microbiome in PTLDS was found to differ from healthy controls (FIG. 8 and Table 8).

TABLE 8
Gut microbiome composition in PTLDS and healthy cohorts

| | Relative | Relative |
|---|---|---|
| | Abundance in | abundance in |
| | PTLDS | Healthy Control |
| Genus | (%) | (%) |
| Akkermansia | 2.740 | 1.002 |
| Alistipes | 1.344 | 6.079 |
| Bacteroides | 14.404 | 24.545 |
| Barnesiella | 0.256 | 1.069 |
| Bifidobacterium | 2.853 | 4.084 |
| Blautia | 14.521 | 4.835 |
| Catenibacterium | 0.010 | 1.747 |
| Clostridium | 5.412 | 4.868 |
| Collinsella | 2.171 | 0.221 |
| Coprococcus | 0.000 | 1.025 |
| Dialister | 1.245 | 1.940 |
| Dorea | 1.486 | 0.764 |
| Enterococcus | 2.857 | 0.000 |
| Escherichia | 4.334 | 0.249 |
| Eubacterium | 6.204 | 9.650 |
| Faecalibacterium | 7.041 | 8.720 |
| Lactobacillus | 1.497 | 0.171 |
| Methanobrevibacter | 0.459 | 2.780 |
| Parabacteroides | 1.098 | 2.900 |
| Prevotella | 0.026 | 5.150 |
| Ruminococcus | 4.057 | 4.030 |
| Shigella | 2.727 | 0.283 |
| Streptococcus | 3.542 | 0.310 |
| Subdoligranulum | 4.437 | 2.090 |
| Other | 15.270 | 11.480 |

The Analysis of Composition of Microbiomes (ANCOM) tool was used to determine which genera were significantly different in PTLDS compared to healthy controls (Mandal et al., Microb Ecol Health Dis. 2015 May 29, 26:27663, the entire contents of which are herein incorporated by reference). ANCOM compares the abundance of one taxon between samples by computing Aitchison's log-ratio of abundance for each taxon relative to the abundance of all other taxa individually. If there are “y” number of taxa, then there are “y−1” tests performed for each taxon; the significance of these tests is calculated using the Benjamini-Hochberg procedure. ANCOM then counts the number of tests that are rejected for each taxon to obtain a count random variable W which represents the number of nulls among the tests that are rejected. The empirical distribution of W determines the final significance of each taxon. This analysis found that a decrease in Bacteroides and increase in Blautia were most significant between the PTLDS and healthy patient groups. Other members of the Bacteroidales order namely Parabacteroides and Bamesiella were significantly decreased in PTLDS, as well as the anti-inflammatory genus Faecalibacterium. In addition to Blautia, the genera Enterococcus and Escherichia were significantly increased in PTLDS (Table 9). In Table 9, genera that are increased by average relative abundance in PTLDS compared to healthy control appear in boldface.

TABLE 9
Significantly different genera in
PTLDS versus control population

| Genus | W Statistic |
|---|---|
| Bacteroides | 158 |
| [symbol] | 157 |
| Alistipes | 156 |
| Eubacterium | 154 |
| [symbol] | 154 |
| Bifidobacterium | 150 |
| Parabacteroides | 150 |
| Faecalibacterium | 149 |
| [symbol] | 144 |
| [symbol] | 143 |
| Odoribacter | 143 |
| [symbol] | 142 |
| Coprococcus | 140 |
| Barnesiella | 135 |
| [symbol] | 134 |
| Bilophila | 134 |
| [symbol] | 134 |
| Roseburia | 134 |
| Butyricimonas | 133 |
| [symbol] | 132 |

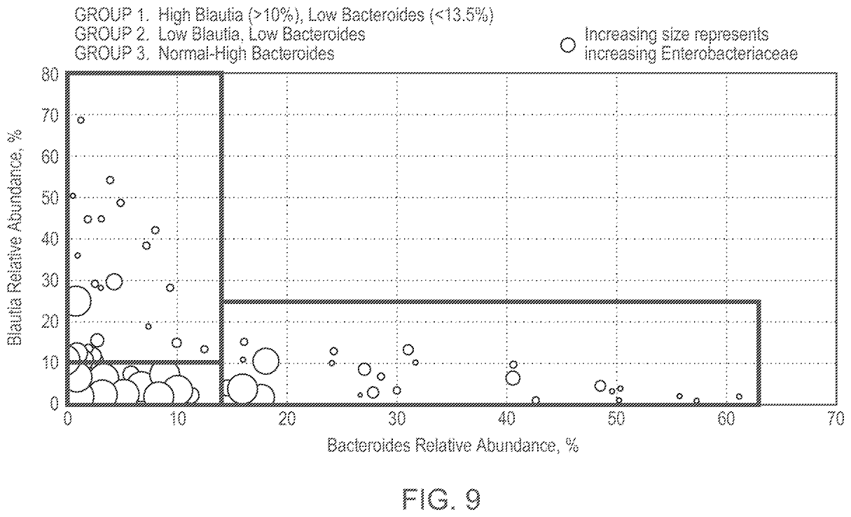

Given the significant reduction in Bacteroides and increase in Blautia, low Bacteroides was gated as being half of the average relative abundance of a subset of approximately 15,000 samples from the American Gut Project, a citizen science project analyzing gut microbiome samples, at 13.5%. High Blautia was gated at four times the American Gut Project average at 10%. These gates were used to define groups: Group 1 having high Blautia (>10%) and low Bacteroides (<13.5%), Group 2 having low Blautia (<10%) and low Bacteroides (<13.5%), and Group 3 having high Bacteroides (<13.5%). Thirty of the 90 PTLDS patients had a microbiome profile of high Blautia (>10%) and low Bacteroides (<13.5%) and were characterized as Group 1. Patients who did not have high Blautia but who had low Bacteroides (<13.5%, Group 2) tended to have increased Enterobacteriaceae, a pathogenic family of bacteria (>10%) (FIG. 9).

For 16s rRNA gene sequencing studies using Illumina technology, a healthy control cohort from the American Gut Project (AGP) (n=158), a citizen science project that has analyzed the gut microbiome of nearly 20,000 individuals, and a control cohort of patients in the ICU who have received lengthy antibiotic regiments from the Extreme Dysbiosis of the Microbiome in Critical Illness study (ICU) (n=128) were analyzed. The ability of the sequence studies to predict PTLDS was investigated. Receiver operating characteristic curve with reported AUC (area under the curve) values evaluating the ability to distinguish PTLDS (SLICE HI) from a healthy subset of the AGP and an AGP ICU cohort based on the fecal microbiome signature were prepared. The AUC values for PTLDS cohort was 0.95, whereas the AUC value for the healthy subset of AGP and AGP ICU was 1.00. This suggests that the microbiome signature is able to predict patients with PTLDS with high accuracy.

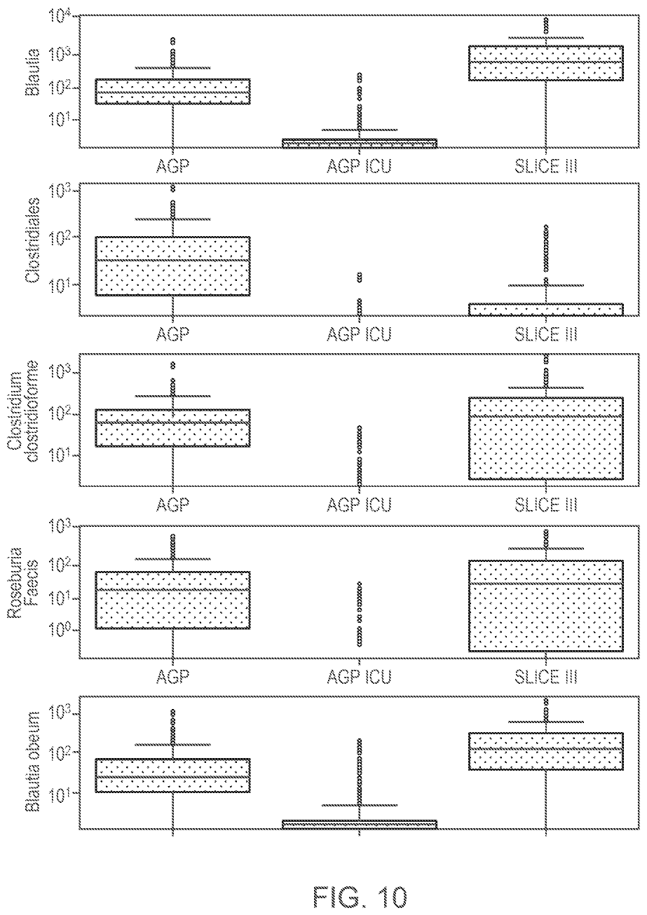

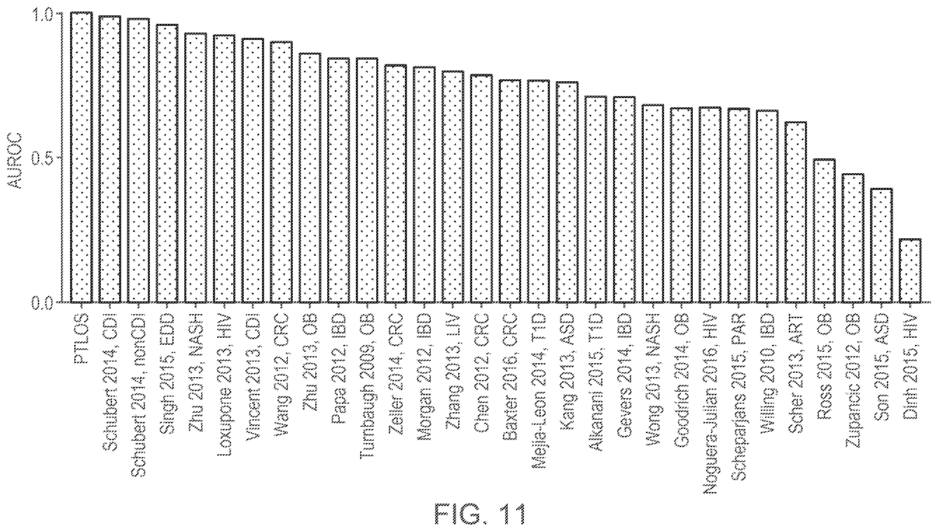

Abundance boxplots of the five most important features that distinguish the fecal microbiome in PTLDS from the AGP healthy and ICU cohorts. In congruence with the differential abundance, the top 5 most predictive features associated with PTLDS are in the Lachnospiraceae family, including the genus Blautia and the species Blautia obeum (FIG. 10).

### Example 6. Comparative Analysis of Microbiome of PTLDS Subjects with Healthy Subjects

Curation of the PTLDS and Control Cohorts

The PTLDS cohort is part of the Study of Lyme disease Immunology and Clinical Events (SLICE) curated at the Johns Hopkins Lyme Disease Research Center. Detailed enrollment and eligibility criteria for this cohort have been previously described in Rebman et al. (2017), Front Med 4:224 (the entire contents of which are herein incorporated by reference). Patients with PTLDS had medical record documentation of prior Lyme disease meeting the CDC surveillance case definitions with appropriate treatment and had current patient-reported symptoms of fatigue, cognitive dysfunction, and/or musculoskeletal pain resulting in functional impairment. Many of those enrolled had received subsequent antibiotics for treatment of their persistent symptoms, and participants were permitted to be actively taking antibiotics for their condition at the time of enrollment. Patients had all received appropriate antibiotic treatment at the time of their initial diagnosis of Lyme disease, and many had received subsequent antibiotics for treatment of persistent symptoms. The median time from Lyme disease symptoms onset to the study visit was 1.1 years (interquartile range [IQR], 0.5 years to 3.3 years), and participants reported taking a median of 56 days (IQR, 30 days to 84 days) of antibiotics during that interval. Eight (9.2%) reported currently taking antibiotics at the time of the study visit. The mean age of this cohort sample was 48.3 years (standard deviation [SD], 14.7), and 36 (41.4%) of the subjects were female. Patients with PTLDS were also excluded for a range of preexisting or comorbid conditions with significant PTLDS symptom overlap and/or immunosuppressive effects. Information on appropriate antibiotic treatment for Lyme disease was abstracted from the medical record; subsequent antibiotic use was recorded as part of the research study visit.

Fecal samples were collected from 87 patients with well-defined PTLDS in the SLICE cohort. Subjects were provided with stool collection containers containing 9 ml of 20% glycerol and BBL culture swabs (Becton, Dickinson and Company, Sparks, Md.). From a single stool sample produced at any time of day, stool was self-collected into the collection container to reach 10 ml and swabs were taken; samples were returned to the Johns Hopkins Lyme Disease Research Center (MD) and stored at −80° C. Samples in stool collection containers were sequenced using Ion Torrent technology, and swabs were sequenced using Illumina technology.

The healthy control cohort consisted of two healthy populations: a healthy cohort at Northeastern University (IT-Healthy; Boston, Mass.) and 152 donors from a healthy subset of the American Gut Project (AGP Healthy). Sample processing for these cohorts was performed according to Earth Microbiome Project protocols (Gilbert J A et al. BMC Biol 12:69; the contents of which are herein incorporated by reference). Using stool collection vessels (Medline Industries), one fresh stool sample was self-collected from 17 healthy adult donors. Donors were excluded if they were currently taking antibiotics or if they had taken antibiotics for at least 2 weeks at the time of collection. A sample of the stool was immediately placed in 9 ml of oxygen-pre-reduced phosphate buffered saline (PBS) to a total of 10 ml of slurry in a 50-ml collection tube (Fisher Scientific). The stool slurry was quickly homogenized in a Coy anaerobic vinyl chamber (Coy Laboratory Products, Inc.) in 5% hydrogen, 10% CO2, and 85% nitrogen at 37° C. Samples were stored at −80° C. and sequenced using Ion Torrent technology as described below. A healthy subset of the American Gut Project was identified as previously described by MacDonanld D et al. mSystems 3:e00031-18 (the contents of which are herein incorporated by reference in its entirety). 152 samples were randomly selected from the healthy subset. Samples were collected and sequenced.

To control for the generally high levels of antibiotic use that could alter the microbiome in patients with PTLDS, a previously curated cohort of 123 samples of intensive care unit (ICU) patients from two time points was also used as a control. The ICU cohort consists of 123 samples from two time points (within 48 h of ICU admission and at ICU discharge or on ICU day 10) from critically ill patients in the intensive care unit in four centers across the United States and Canada. Sample collection and processing for this cohort were performed according to Earth Microbiome Project protocols. This cohort served as a control for the effect of antibiotics on the microbiome, as the ICU patients had omnipresent antibiotic use. All ICU patients were treated with differing antibiotic regimens.

Preparation of DNA and 16S rRNA Sequencing Protocols

DNA extraction and sequencing were performed by MR DNA (Shallowater, Tex.) on an Ion Torrent PGM. The V4 variable region was amplified using PCR described in Morrissette M, et al. 2020. mBio 11:e02310-20 (the contents of which are herein incorporated by reference in its entirety) in a single-step 30 cycle PCR with the HotStarTaq Plus master mix kit (Qiagen, USA). The following conditions were used: 94° C. for 3 min and 30 cycles of 94° C. for 30 s, 53° C. for 40 s, and 72° C. for 1 min, followed by a final elongation step at 72° C. for 5 minutes.

Sequencing was also performed using Illumina using the primers 515f/806rB, the V4 region was amplified and was sequenced using an Illumina MiSeq. Sequencing data for the ICU cohort and the American Gut project were obtained in Qiita (study IDs 2136 and 10317). Raw sequencing data were uploaded and processed in Qiita (study ID 11673); the sequences were demultiplexed and trimmed to 150 bp, and closed-reference OTUs were picked with Greengenes 13-8 on an OTU similarity level of 97%. Closed-reference operational taxonomic units (OTUs), a common designation was used instead of “species” or “genus,” were generated (97% identity) and analyzed. Closed-reference picking was performed because it allowed for increased sample size of the PTLDS cohort due to samples being processed in different platforms, but the conclusions of the study did not from vary from the analysis using a subset of samples sequenced by Illumina technology and processed with Deblur to generate amplicon sequence variants. The OTU table was rarefied to 10,000 reads. Data were subsequently analyzed using the software package QIIME2. Since Ion Torrent and Illumina sequencing both followed the Earth Microbiome Project protocol, the sequencing platform did not have a measurable effect on the data and the results from both the sequencing platforms was combined for analysis. To assess the ability of the PTLDS microbiome to be distinguished from healthy and ICU controls, the sample classifier tool in QIIME2 was used. A random-forest classifier was trained and evaluated. ROC curves were generated to summarize the true-versus false-positive rates; the area under the curve was calculated and reflects the ability of the classifier to distinguish between cohorts. The top five most important features for distinguishing the microbiomes were reported. Data generated in this study are available in Qiita (11673) and the European Bioinformatics Institute (ERP122507).

### Sample Classification of PTLDS, ICU, and Healthy Fecal Microbiomes

The ability of the fecal microbiome to distinguish PTLDS, ICU, and healthy cohorts was evaluated using a supervised-learning random-forest classifier model to classify sample cohorts. QIME2 classifier model pipeline was implemented. First, the 16S rRNA gene sequencing data was labelled by cohort. This served as the input for the pipeline. The data was split into two samples: (a) training sample set (b) test sample set. The training sample set was used to train and optimize a random forest classifier model. This method was used to identify important features which were used to predict disease state in test samples and evaluate the model. The information from the training samples was then applied to a test sample set. ROC curves and confusion matrices were generated to evaluate the model.

Receiver operating characteristic (ROC) analysis was used to evaluate the accuracy of the model's classifications. The model's performance was quantified by reducing the two-dimensional ROC curve into a one-dimensional scalar value, i.e., the AUROC as defined above. An AUROC is a value between 0 and 1, where 0.5 would lie along the diagonal line and indicate that the model was as effective at classifying samples as random chance. Higher AUROC values are indicative better model predictions. The model generated herein robustly distinguished the three cohorts with high accuracy, yielding rounded AUROC values of 1, which indicates strong differences in the microbiome of these cohorts. ICU samples versus healthy or PTLDS samples were correctly classified in 100% of samples, which was expected, given the heavy use of antibiotics in the ICU patient group which typically result in severe alteration of the microbiome. The model also classified 82.4% of PTLDS samples against ICU and healthy controls, whereas only 17.6% of PTLDS samples were misclassified as healthy (see Table 10).

TABLE 10
Probability of distinguishing PTLDS from healthy and
ICU controls based on the fecal microbiome composition

| | Value for cohort | | |
|---|---|---|---|
| Cohort | ICU | PTLDS | Healthy |
| ICU | 1 | 0 | 0 |
| PTLDS | 0 | 0.824 | 0.176 |
| Healthy | 0 | 0 | 1 |

The relative abundance of the five most important features (OTUs) for sample classification were identified using QIIME2. Of note, Blautia species (OTU identifiers [IDs]4474380, 4465907, and 4327141) included three of the five important features for classification and were observed at a significantly greater relative abundance in the PTLDS cohort (8.86%±1.26%) than in the ICU (0.070%±0.017%) or healthy (1.34%±0.18%) cohort (P value≤0.0001 for all cohorts versus each other). Conversely, the two other top five features most important for classification were Staphylococcus aureus (OTU ID 446058), which was present at a significantly higher relative abundance in the ICU cohort (0.95%±0.56%) (P value≤0.0001) than in the PTLDS (0.0024%±0.00030%; albeit non-significant) or healthy (0.0077%±0.0020%) cohort, likely due to it being a widespread nosocomial pathogen, and a Roseburia species (OTU ID 4481427) elevated in the healthy cohort (0.29%±0.050) compared to PTLDS (0.15%±0.045) (not significant [NS]) or ICU (0.0024%±0.0013) (P value≤0.0001).

### Example 7: Effect of Antibiotics on Patients with PTLDS

Patients with PTLDS may have been treated with antibiotics such as amoxicillin or doxycycline to curb Borrelia burgdorferi. In some instances, the treatment is not effective in eliminating the Borrelia. However, treatment with antibiotics can alter microbiome composition. The effect of antibiotics on the distinctive microbiome observed in PTLDS was examined. Table 11 provides the summary of the antibiotics used within the PTLDS cohort. In Table 11, time refers to the period within which antibiotics were taken prior to sample donation. Antibiotic use as described in Table 11, is the total number of patients (percent) who have used antibiotics within the time frame. Doxycycline and amoxicillin columns describe the number and percentage of PTLDS patients who have taken doxycycline and/or amoxicillin during the indicated time frame.

TABLE 11
Summation of antibiotic use within PTLDS cohort

| | No. (%) for: | | |
|---|---|---|---|
| Time | Antibiotic use | Doxycycline | Amoxicillin |
| 1 week | 23 (26.4) | 6 (6.9) | 12 (13.8) |
| 1 month | 36 (41.4) | 12 (13.8) | 15 (17.2) |
| 6 month | 64 (73.6) | 35 (40.2) | 19 (21.8) |
| 1 year | 79 (90.8) | 46 (52.9) | 24 (27.6) |

To investigate the role antibiotics may play in shaping the microbiome of patients with PTLDS, principal-coordinate analysis was used to identify the type of antibiotic used and the time since last antibiotic use relative to when the stool sample was collected. Importantly, patients with PTLDS did not cluster by time since antibiotic treatment (1 week, 1 month, 6 months, 1 year, or over 1 year) or by type of antibiotic (doxycycline, amoxicillin, both, other, or none) in principal-coordinates analysis (see Table 12). The PTLDS cohort was then separated into groups based on how recently a patient had taken antibiotics, i.e., within 1 week to 1 month or greater than or equal to 6 months, and used a supervised random-forest classifier model to evaluate the ability of antibiotic history to distinguish these groups within the PTLDS cohort and healthy and ICU samples. The difference in antibiotic administration regimens did not distinguish patients within the PTLDS cohort (Table 12). These results suggest that antibiotic influence alone cannot explain the distinctive microbiomes of PTLDS patients when compared to other cohorts.

TABLE 12
AUROC of a random-forest classifier model to
classify the fecal microbiome in different cohorts

| Group | AUROC value |
|---|---|
| PTLDS: Last | 0.92 |
| antibiotic use 1 week- | |
| 1 month | |
| PTLDS: Last | 0.96 |
| antibiotic use≥ | |
| ICU | 0.99 |
| Healthy | 1.00 |

### Example 8: Subclassification of Patients with PTLDS

To further study the microbiome in PTLDS patients, patients were sub-grouped based on important taxonomic features. The genus Blautia which was the most represented genus in the classification was used for further subclassification. PTLDS patients with a relative abundance of Blautia of over 10% tended to have a decreased abundance of the genus Bacteroides, (below 15%) compared to an average relative abundance of 23.15% in the healthy cohort. The relative abundance of Bacteroides in the fecal microbiome of patients with PTLDS was significantly lower (P value≤0.0001) than healthy cohort controls. Statistical significance was determined using the Kruskal-Wallis (nonparametric) test followed by Dunn's multiple comparison. The relative abundance of Bacteroides in the fecal microbiome of ICU patients with PTLDS was also significantly lower than the healthy cohort (P value≤0.001), but to a lesser extent than the PTLDS cohort versus healthy cohort.

Bacteroides was used as a secondary grouping metric. The importance of Bacteroides as a common gut symbiont and the correlation between decreased Bacteroides in diseases with symptoms overlapping those of PTLDS, such as depression provided strong rationale for using Bacteroides as the secondary grouping metric. Plotting the relative abundance of Bacteroides versus the relative abundance of Blautia yielded three distinct subgroups in the PTLDS cohort, which we defined are defined herein as group 1 (G1)>10% Blautia and <15% Bacteroides; group 2 (G2), >15% Bacteroides and group 3 (G3)<10% Blautia and <15% Bacteroides. Few samples in the healthy (1.78% of samples) and ICU (0.813%) cohorts overlapped with G1 (high Blautia and low Bacteroides), which included 30.92% of PTLDS samples, but greater overlap existed in G2 and G3. In the classifier model, all samples in the test set that were defined as G1 were correctly classified as PTLDS.

Expansion of proinflammatory Enterobacteriaceae is a common feature of disease associated microbiomes. The relative abundance of Enterobacteriaceae in patients with PTLDS was examined. Although the family Enterobacteriaceae was not in the top 5 most important features for classification of the microbiome in PTLDS, healthy, and ICU cohorts, some patients with PTLDS had exceptionally high levels of Enterobacteriaceae compared to the healthy control population at Northeastern University (IT-Healthy). Of the 193 OTUs in the Enterobacteriaceae family represented in the combined data sets (PTLDS, ICU, and healthy) in this study, the mean relative abundance of Enterobacteriaceae in IT-Healthy was 1.14% (median±0.0275%), which was significantly lower than the average relative abundance of 9.20% in PTLDS subjects (median±0.46%) (P value≤0.01; Kruskal-Wallis (nonparametric) test followed by Dunn's multiple comparison). Approximately one-fifth (19.5%) of patients with PTLDS presented with a relative abundance of Enterobacteriaceae over 10%. As expected, ICU patients had a higher average relative abundance of Enterobacteriaceae (31.21%) when compared to both PTLDS patients (P value≤0.0001) and the healthy cohort (P value≤0.0001).

### Example 9: Microbiome as a Disease State Predictor

Microbiome-associated studies (MAS) have been found to be excellent predictors in various diseases, often outperforming genome-wide association studies, likely because the microbiome is a confluence of genetics and the environment. ROC analysis of the PTLDS cohort yielded a rounded AUROC of 1.00, correctly classifying patients with PTLDS for 82.4% of samples. These results are similar in accuracy to results for well-established microbiome-associated diseases such as Clostridium difficile infection, while outperforming the predictive capabilities of other MAS, such as IBD, in which abnormalities within the microbiome are strongly implicated (Duvallet et al. (2017), Nat. Commun. 8:1784, the entire contents of which are herein incorporated by reference) (see Table 13 and FIG. 11).

TABLE 13
Ranked area under receiver
operating characteristic curve (AUROC) reported
by Duvallet et al. (2017), Nat. Commun. 8:1784

| Author date, disease | AUROC |
|---|---|
| Singh 2015, EDD | 0.96 |
| Schubert 2014, CDI | 0.99 |
| Schubert 2014, nonCDI | 0.98 |
| Vincent 2013, CDI | 0.91 |
| Goodrich 2014, OB | 0.67 |
| Turnbaugh 2009, OB | 0.84 |
| Zupancic 2012, OB | 0.44 |
| Ross 2015, OB | 0.49 |
| Zhu 2013, OB | 0.86 |
| Baxter 2016, CRC | 0.77 |
| Zeller 2014, CRC | 0.82 |
| Wang 2012, CRC | 0.9 |
| Chen 2012, CRC | 0.78 |
| Gevers 2014, IBD | 0.71 |
| Morgan 2012, IBD | 0.81 |
| Papa 2012, IBD | 0.84 |
| Willing 2010, IBD | 0.66 |
| Noguera-Julian 2016, HIV | 0.67 |
| Lozupone 2013, HIV | 0.92 |
| Dinh 2015, HIV | 0.22 |
| Son 2015, ASD | 0.39 |
| Kang 2013, ASD | 0.76 |
| Alkanani 2015, T1D | 0.71 |
| Mejia-Leon 2014, T1D | 0.77 |
| Wong 2013, NASH | 0.68 |
| Zhu 2013, NASH | 0.93 |
| Scher 2013, ART | 0.62 |
| Zhang 2013, LIV | 0.8 |
| Scheperjans 2015, PAR | 0.67 |

### EQUIVALENTS AND SCONE

Those skilled in the art will recognize, or be able to ascertain using no more than routine experimentation, many equivalents to the specific embodiments in accordance with the disclosure described herein. The scope of the present disclosure is not intended to be limited to the above description, but rather is as set forth in the appended claims. It should be understood, for example, that via whole genome sequencing and annotation, various bacteria with the genetic capability to respire anaerobically can be identified to reduce the bloom of Enterobacteriaceae and cyclic inflammation in patients with PTLDS and/or inflammatory conditions. These bacteria can be tested to reduce Enterobactericeae in a human gut simulator, with a microbiome community derived from patients with PTLDS or other inflammatory conditions. These bacteria can be further tested for limited or anti-inflammatory effects in models such as the LPS-induced inflammatory assay in human peripheral blood mononuclear cells (PBMC).

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed. The term “consists essentially of” means excluding other materials that contribute to function or structure. For example, a composition consisting essentially of a pharmaceutically active ingredient may include other ingredients, such as excipients, that do not affect the function or structure of the active ingredient. Percentages refer to weight percentages unless otherwise indicated.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A composition for inhibiting growth of at least one species of Enterobacteriaceae, comprising a bacterial population of at least one bacterial species selected from the group consisting of Gordonibacter pamelaeae, Clostridium bifermentans, Veillonella ratti, Paraclostridium benzoelyticum, Sutterella wadsworthia, Alisteps onderdonkii, Barnesiella intestinihominis, Clostridium hathewayi, Bifidobacterium catenulatum, Anaerinibacillus anaerinlyticius, Coprobacillus catenformis, or Coprococcus comes.

2. The composition of claim 1, wherein the composition consists essentially of a bacterial population of three bacterial species.

3. The composition of claim 2, wherein the three bacterial species are Gordonibacter pamelaeae, Clostridium bifermentans, and Veillonella ratti.

4. The composition of claim 2, wherein the three bacterial species are present in the bacterial population in a ratio of 2:1:1.

5. The composition of claim 2, wherein the three bacterial species are present in the bacterial population in a ratio of 1:1:1.

6. The composition of claim 1, wherein the composition comprises at least 1×10⁸ colony-forming units (CFU) of the bacterial population.

7. The composition of claim 1, wherein the composition inhibits the growth of at least one species of Enterobacteriaceae by from about 1 fold to about 20 fold.

8. (canceled)

9. The composition of claim 1, wherein the composition comprises a bacterial population of at least one bacterial species selected from the group consisting of Gordonibacter pamelaeae, Clostridium bifermentans and Veillonella ratti.

10-13. (canceled)

14. The composition of claim 1, wherein the at least one species of Enterobacteriaceae is Escherichia coli.

15. The composition of claim 1, wherein the composition is in a form of a drug, a pharmaceutical preparation including at least one pharmaceutically acceptable carrier, a probiotic, a prebiotic, a capsule, a tablet, a caplet, a pill, a troche, a lozenge, a powder, a granule, a dietary ingredient, a food, a food supplement, a medical food, or a combination thereof.

16. A method of treating post-treatment Lyme disease syndrome in a subject, comprising:
contacting the subject with or administering to the subject the composition of claim 1; and
inhibiting the growth of a least one species of Enterobacteriaceae.

17. A method of treating or preventing inflammation in a subject, comprising
contacting the subject with the composition of claim 1; and
inhibiting the growth of at least one species of Enterobacteriaceae.

18-22. (canceled)

23. The method of claim 16, wherein the composition is administered to the subject via at least one of an oral route, a buccal route, a subcutaneous route, an intravenous route, an intramuscular route, an intraperitoneal route, a transdermal route, an ocular route, a vaginal route, a nasal route, or a topical route.

24. The method of claim 23, wherein the composition is administered by the oral route.

25. The method of claim 16, wherein the composition is provided to the subject in an amount of about 1×10⁸ colony-forming units of the bacterial population per kg body weight of the subject.

26. A method of diagnosing post-treatment Lyme disease syndrome (PTLDS) in a subject comprising:
a. obtaining a sample from the subject;
b. measuring a relative abundance of one or more bacterial genera in the sample to prepare a microbiome signature, the bacterial genera being at least one of Blautia, Clostridium, Roseburia, Staphylococcus, Bacteroides Parubacteroides, Barnesiella, Faecalibacterium, Enterococcus, Escherichia, Akkermansia, Alistipes, Barnesiella, Bifidobacterium, Catenibacterium, Collinsella, Coprococcus, Dialister, Dorea, Eubacterium, Lactobacillus, Methanobrevibacter, Prevotella, Ruminococcus, Shigella, Streptococcus, or Subdoligranulum; and
c. comparing the microbiome signature of the sample to a microbiome signature of a healthy control cohort,
wherein a difference in the relative abundance of one or more bacterial genera in the microbiome signature of sample compared to the microbiome signature of the healthy control cohort confirms the presence of PTLDS in the subject.

27. The method of claim 26, wherein the sample from the subject is a stool sample.

28. The method of claim 26, wherein 16S rRNA is extracted from the sample prior to measuring the levels of one or more bacterial genera in the sample and wherein levels of the one or more bacterial genera in the sample is measured by 16S rDNA gene sequencing.

29. (canceled)

30. The method of claim 26, wherein the one or more bacterial genera comprises Blautia and wherein the Blautia is Blautia obeum.

31. (canceled)

32. The method of claim 30, wherein Blautia consists essentially of one or more Operational Taxonomic Units IDs selected from 4474380, 4465907, or 4327141.

33. The method of claim 30, wherein a relative abundance level of Blautia in the sample is greater than the healthy control cohort and wherein the wherein the relative abundance level of Blautia in the sample is from about 5% to about 10%.

34-35. (canceled)

36. The method of claim 26, wherein the one or more bacterial genera comprise Staphylococcus and wherein the Staphylococcus is Staphylococcus aureus.

37. (canceled)

38. The method of claim 36, wherein Staphylococcus consists essentially of an Operational Taxonomic Unit ID 446058.

39. The method of claim 36, wherein the relative abundance level of Staphylococcus in the sample is greater than the healthy control cohort and wherein the relative abundance level of Staphylococcus in the sample is from about 0.001% to about 0.1%.

40-41. (canceled)

42. The method of claim 26, where in the one or more bacterial genera is Roseburia and wherein Roseburia consists essentially of an Operational Taxonomic Unit ID 4481427.

43. (canceled)

44. The method of claim 42, wherein a relative abundance level of Roseburia in the sample is greater than the healthy control cohort and wherein the relative abundance level of Roseburia in the sample is from about 0.10% to about 0.20%.

45. (canceled)

46. The method of claim 42, wherein the relative abundance level of Roseburia in the sample is 0.15%.

47. The composition of claim 1,
wherein the composition is a non-drug product and wherein the non-drug product is a dietary ingredient, a food, a food supplement, a medical food, or a combination thereof.

48-61. (canceled)

62. The method of treating post-treatment Lyme disease syndrome in a subject according to claim 16, wherein in the at least one species of Enterobacteriaceae is Escherichia coli.

63. The method of treating post-treatment Lyme disease syndrome in a subject according to claim 16, the composition inhibits the growth of at least one species of Enterobacteriaceae by from about 1 fold to about 20 fold.
